(19) United States
(12) Patent Application Publication
(10) Pub. No.: US 2021/0207101 A1
(43) Pub. Date: Jul. 8, 2021

(54) Hybrid Thymus, Methods of Making, and Methods of Using to Induce Xenograft Tolerance, Restore Immunocompetence and Thymic Function

(71) Applicant: THE TRUSTEES OF COLUMBIA UNIVERSITY IN THE CITY OF NEW YORK, New York, NY (US)

(72) Inventors: Megan Sykes, New York, NY (US); Mohsen Khosravi-Maharlooei, New York, NY (US); Nichole Danzl, New York, NY (US)

(21) Appl. No.: 17/204,147

(22) Filed: Mar. 17, 2021

Related U.S. Application Data
(63) Continuation of application No. PCT/US2019/051865, filed on Sep. 19, 2019.
(60) Provisional application No. 62/734,019, filed on Sep. 20, 2018.

(51) Int. Cl. C12N 5/071; C12N 5/078; A61L 27/38; A61L 27/36

(52) U.S. Cl. CPC C12N 5/0697; C12N 5/065; A61L 27/3813; C12N 2506/02; A61L 27/3604; C12N 2506/45; A61L 27/3695

(57) ABSTRACT

The present disclosure relates to making a hybrid pig-human thymic tissue and using the hybrid thymic tissue to induce tolerance in xenotransplantation. The hybrid thymic tissue can also be used for restoring or inducing immunocompetence in a recipient as well as restoring or promoting thymus-dependent ability for T cell progenitors to develop into mature functional T cells in a recipient.

### CROSS-REFERENCE TO OTHER APPLICATIONS

The present application is a continuation of PCT/US2019/051865, filed Sep. 19, 2019, which claims priority to U.S. Patent Application Ser. No. 62/734,019 filed Sep. 20, 2018, all of which are incorporated by reference, as if expressly set forth in their respective entireties herein.

### STATEMENT OF GOVERNMENTAL INTEREST

This invention was made with government support under AI084903, AI045897, and AI106697 awarded by the National Institutes of Health. The government has certain rights in the invention.

### FIELD OF THE INVENTION

The present disclosure relates to making a pig-human hybrid thymus and using the hybrid thymus to induce tolerance in xenotransplantation.

### BACKGROUND OF THE INVENTION

The severe shortage of allogeneic donors currently limits the number of organ transplants performed. This supply-demand disparity may be corrected by the use of organs from other species (xenografts). In view of the ethical issues and impracticalities associated with the use of non-human primates, pigs are considered the most suitable donor species for humans. In addition to organ size and physiologic similarities to humans, the ability to rapidly breed and inbreed pigs makes them particularly amenable to genetic modifications that could improve their ability to function as graft donors to humans. Sachs, Path. Biol. 42:217-219, 1994; Piedrahita et al., Am. J. Transplant, 4 Suppl. 6:43-50, 2004.

Despite recent advances, the immune response to xenografts remains powerful, limiting their clinical use. Although transplantation coupled with non-specific immunosuppressive therapy is associated with high early graft tolerance, a major limitation to the success of clinical organ transplantation has been late graft loss, due largely to chronic rejection of the transplant. Furthermore, immunosuppressive therapies often carry adverse side effects or increase risk of infection. As such, a method that controls the immune response to xenografts would greatly improve their applicability.

Genetically engineered pigs lacking the Gal gene avoid common rejection in non-human primates owing to anti-Galα1-3Gal (Gal) natural antibodies. Cooper D. A brief history of cross-species organ transplantation. Proc (Bayl Univ Med Cent). 2012 January; 25(1): 49-57. Despite this advancement, T cell-dependent antibodies can recognize other pig specificities causing rejection. Yang Y G, Sykes M. Xenotransplantation: current status and a perspective on the future. Nat Rev Immunol. 2007 July; 7(7):519-31. T cell-suppression prolonged porcine xenograft survival in nonhuman primates, but such treatments are highly toxic. Yamada K, Sykes M, Sachs D H. Tolerance in xenotransplantation. Curr Opin Organ Transplant. 2017 December; 22(6):522-528.

An alternative approach to T-cell suppression is tolerance induction. The xenograft tolerance approaches include mixed chimerism induction and porcine thymic transplantation.

Mixed chimerism can induce tolerance to the donor at the level of T cells, B cells and natural killer (NK) cells in the recipient. Griesemer A., Yamada K. and Sykes M., Xenotransplantation: Immunological hurdles and progress toward tolerance, Immunol. Rev. 2014; 258(1): 241-258. Sachs D. H., Kawai T. and Sykes M., Cold Spring Harb. Perspect. Med. 2014; 4:a015529.

Thymic xenotransplantation can also induce robust tolerance. Kalscheuer H, Onoe T, Dahmani A, Li H W, Hölzl M, Yamada K, Sykes M. Xenograft Tolerance and Immune Function of Human T Cells Developing in Pig Thymus Xenografts. J Immunol. 2014 Apr. 1; 192(7):3442-50. Porcine thymic grafts can generate diverse and functional human T cell repertoires in mice that are specifically unresponsive to the donor pig in vitro. Shimizu I, Fudaba Y, Shimizu A, Yang Y, and Sykes M. Comparison of human T cell repertoire generated in xenogeneic porcine and human thymus grafts. Transplantation. 2008 Aug. 27; 86(4): 601-610. Nikolic B., J. P. Gardner, D. T. Scadden, J. S. Arn, D. H. Sachs, M. Sykes. Normal development in porcine thymus grafts and specific tolerance of human T cells to porcine donor MHC. J. Immunol. 1999 Mar. 15; 162: 3402-3407. Habiro K, Sykes M, Yang Y G. Induction of human T-cell tolerance to pig xenoantigens via thymus transplantation in mice with an established human immune system. Am J Transplant. 2009 June; 9(6):1324-9. Extension of this approach from the pig to the baboon model has achieved lasting pig kidney xenograft survival in nonhuman primates. Yamada K, Yazawa K, Shimizu A, Iwanaga T, Hisashi Y, Nuhn M, O'Malley P, Nobori S, Vagefi P A, Patience C, Fishman J, Cooper D K, Hawley R J, Greenstein J, Schuurman H J, Awwad M, Sykes M, Sachs D H. Marked prolongation of porcine renal xenograft survival in baboons through the use of alpha1,3-galactosyltransferase gene-knockout donors and the cotransplantation of vascularized thymic tissue. Nat Med. 2005 January; 11(1):32-4.

However, remaining limitations include a suboptimal recipient's immune function that fails to recognize efficiently foreign antigens; suboptimal survival, homeostasis and inefficient removal of autoreactive T cells; and lack of positive selection of regulatory T cells to prevent autoimmunity.

### SUMMARY OF THE INVENTION

The present disclosure provides for a method of inducing tolerance in a recipient mammal of a first species to a graft obtained from a donor mammal of a second species, the method comprising the steps of:

(a) introducing a hybrid thymic tissue into the recipient mammal, wherein the hybrid thymic tissue is a thymic tissue from the second species and comprises thymic epithelial cells from the first species; and

(b) implanting the graft from the donor mammal in the recipient mammal.

In some embodiments, the thymic function is essentially absent in the recipient mammal before performing step (a). In some embodiments, the recipient mammal is a primate and in some embodiments, it is a human. In some embodiments, the donor mammal is a swine and in some embodiments, it is a miniature swine.

In some embodiments, the thymic tissue from the donor mammal is a fetal thymic tissue. In some embodiments, the thymic tissue from donor mammal is a neonatal thymic tissue.

In some embodiments, the thymic epithelial cells from the recipient mammal are obtained from the recipient mammal's thymus. In some embodiments, the thymic epithelial cells from the recipient mammal are generated from the recipient mammal's induced pluripotent stem cells (iPSCs). In some embodiments, the thymic epithelial cells from the recipient mammal species are generated from embryonic stem cells that share HLA alleles with the recipient mammal. In some embodiments, the embryonic stem cells are genetically engineered to share HLA alleles with the recipient mammal.

In some embodiments, the hybrid thymic tissue is implanted in the recipient mammal in step (a). In some embodiments, step (a) is conducted prior to, or simultaneous with, step (b).

In some embodiments, the hybrid thymic tissue is generated by introducing thymic epithelial cells from the recipient mammal into the thymic tissue from the donor mammal. In some embodiments, the hybrid thymic tissue is generated by injecting thymic epithelial cells from the recipient mammal of the first species into the thymic tissue from the donor mammal of the second species. In some embodiments, the method further includes administering hematopoietic stem cells (HSCs) to the recipient mammal. In some embodiments, the graft comprises cells, a tissue or an organ.

In further embodiments, the hybrid thymic tissue is generated by a method comprising the following steps:

(i) treating the thymic tissue from the donor mammal of the second species with 2-deoxyglucose (2DG); and

(ii) introducing thymic epithelial cells from the recipient mammal of the first species into the 2DG-treated thymic tissue.

In some embodiments, the thymic epithelial cells from the first species are suspended in Matrigel before being injected into the 2DG-treated thymic tissue.

The disclosure also provides for a method restoring or inducing immunocompetence in a recipient mammal of a first species, the method comprising the step of introducing a hybrid thymic tissue into the recipient mammal of the first species, wherein the hybrid thymic tissue is a thymic tissue from a donor mammal of a second species and comprises thymic epithelial cells from the first species.

The present disclosure also provides for a method of restoring or promoting thymus-dependent ability for T cell progenitors to develop into mature functional T cells in a recipient mammal of a first species, the method comprising introducing a hybrid thymic tissue into the recipient mammal of the first species, wherein the hybrid thymic tissue is a thymic tissue from a donor mammal of a second species and comprises thymic epithelial cells from the first species.

In some embodiments of these methods, thymic function is essentially absent in the recipient mammal before the introducing step. In some embodiments, the recipient mammal is thymectomized before the introducing step. In some embodiments, the recipient mammal has an immune disorder.

In some embodiments, the donor mammal is a swine and in some embodiments, the swine is a miniature swine. In some embodiments, the recipient mammal is a primate. In some embodiments, the recipient mammal is a human.

In some embodiments, the thymic tissue from the donor mammal is a fetal thymic tissue. In some embodiments, the thymic tissue from the donor mammal is a neonatal thymic tissue.

In some embodiments, the thymic epithelial cells are obtained from the recipient mammal's thymus. In some embodiments, the thymic epithelial cells are generated from the recipient mammal's induced pluripotent stem cells (iPSCs). In some embodiments, the thymic epithelial cells are generated from embryonic stem cells that share HLA alleles with the recipient mammal. In some embodiments, the embryonic stem cells are genetically engineered to share HLA alleles with the recipient mammal.

In some embodiments, the hybrid thymic tissue is implanted in the recipient mammal.

In some embodiments, the hybrid thymic tissue is generated by introducing thymic epithelial cells from the first species into the thymic tissue from the donor mammal of the second species. In some embodiments, the hybrid thymic tissue is generated by injecting thymic epithelial cells from the first species into the thymic tissue from the donor mammal of the second species.

In some embodiments, the hybrid thymic tissue is generated by a method comprising the following steps:

(i) treating the thymic tissue from the donor mammal of the second species with 2-deoxyglucose (2DG); and

(ii) introducing thymic epithelial cells from the first species into the 2DG-treated thymic tissue.

In some embodiments, the thymic epithelial cells from the first species are suspended in Matrigel before being injected into the 2DG-treated thymic tissue.

The present disclosure also provides for an isolated hybrid thymic tissue comprising thymic epithelial cells from a first mammalian species and a thymic tissue from a second mammalian species and a method for making a hybrid thymic tissue.

In some embodiments, the second mammalian species is a swine and in some embodiments, the swine is a miniature swine. In some embodiments, the first mammalian species is a primate. In some embodiments, the recipient mammal is a human.

In some embodiments, the thymic tissue from the second mammalian species is fetal thymic tissue. In some embodiments, the thymic tissue from the second mammalian species is a neonatal thymic tissue.

In some embodiments, the thymic epithelial cells are obtained from a thymus from a first mammalian species. In some embodiments, the thymic epithelial cells from the first mammalian species are obtained from fetal thymic tissue. In some embodiments, the thymic epithelial cells from the first mammalian species are obtained from neonatal thymic tissue. In some embodiments, the thymic epithelial cells are generated from induced pluripotent stem cells (iPSCs) from the first mammalian species. In some embodiments, the thymic epithelial cells are generated from embryonic stem cells that share HLA alleles with the first mammalian species. In some embodiments, the embryonic stem cells are genetically engineered to share HLA alleles with the first mammalian species.

The present disclosure also provides for an isolated hybrid thymic tissue comprising thymic epithelial cells from a first mammalian species and a thymic tissue from a second mammalian species and a method for making a hybrid thymic tissue, comprising the steps of:

(i) treating a thymic tissue from a second mammalian species with 2-deoxyglucose (2DG); and

(ii) introducing thymic epithelial cells from a first mammalian species into the 2DG-treated thymic tissue.

In some embodiments, the thymic epithelial cells from the first mammalian species are suspended in Matrigel before being injected into the 2DG-treated thymic tissue.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1. Generation of human/pig hybrid thymus. At 12-20 weeks post-transplantation, humanized mice generated with hybrid pig/human thymus and human CD34+ cells were euthanized and the grafted thymi removed, sectioned and stained to detect human TECs using two-photon confocal microscopy. FIGS. 1A, 1B and 1C show images of the grafted pig thymus uninjected (FIG. 1A), injected with human fetal thymic stromal cells (gestational age of 20 weeks) (FIG. 1B), and injected with pediatric human thymic stromal cells (from a 4-month-old thymus) (FIG. 1C). Arrows in B and C point to CK14+HLA-DR+ cells, which represent human TECs inside pig thymus. Quantitative analysis of entire sections shown in FIGS. 1A-1C is shown in FIG. 1D. Additional controls of human fetal and pediatric thymus and pig thymus were also included in quantitative analysis. The analysis was performed by lmarisColoc software, which allowed the calculation of colocalization of CK14+HLA-DR+ cells among all CK14+ cells in entire thymic sections. Numbers shown on top of the bars are percentages of CK14+HLA-DR+ cells among CK14+ cells. FIGS. 1E and 1F show representative images of the expanded human thymic mesenchyme cells (TMCs) (FIG. 1E) and TECs (FIG. 1F), which were cultured on a 3-D Matrigel culture system for 3 weeks from huCD45-depleted human thymic cells following digestion of a 17-year-old pediatric thymus with liberase. FIGS. 1G and 1H show representative flow cytometric characterization of expanded TMCs and TECs, respectively. CD105-CD326+ are considered as TECs, while CD105+CD326− cells are considered to be TMCs.

FIG. 2. CK14+HLA-DR+ cells were detected in hybrid thymi generated by injecting human thymic stromal cells (from a fetal thymus) (TEC) into the pig thymus. FIG. 2A: no human TEC injection. FIG. 2B: human TEC injected. FIG. 2C: 2-DG-treated pig thymus+human fetal TEC injected.

FIG. 3. Results showing the development of methods for use in generating a hybrid thymus. FIG. 3A shows flow cytometry results showing the number of released cells after injection of cells into the swine fetal thymic fragments. PBMCs cells were resuspended in Matrigel to prevent them from leaking out of the pig thymus after injection. Three different methods were used for injection of the cells into the thawed swine fetal thymic fragments. Method A: Injection using Hamilton syringe while the pieces were placed inside the wells of a V-bottom 96-well plate. Method B: Injection using PESO tubing. Method C: Injection using Hamilton syringe while the pieces were kept outside the well with forceps until the Matrigel is solidified. The number of released cells was determined by flow cytometry to track the CFSE-stained injected PBMCs. FIGS. 3B and 3C shows the results of various reagents to deplete the thymocytes in pig thymic fragments ex vivo. FIG. 3B are graphs of the total live cell count for cells treated with each reagent (top panel) and the percent of live cells (bottom panel) for cells treated for each reagent. FIG. 3C are graphs of the ratio of remaining live double positive CD4 and CD8 cells (DP) or single positive (SP) CD4 (SP-CD4) or SP-CD8 cells on double negative (DN) cells, which contain the stromal cells, used as the readout. Treatment with 2DG 100 nM for 12 hours resulted in the lowest ratios and therefore was the best strategy for removing thymocytes while preserving the stromal cells.

FIG. 4 shows the experimental scheme for preparing hybrid thymi and transplanting the hybrid thymi into recipient mice.

FIG. 5 shows graphs of the levels of human immune cell reconstitution after hybrid thymi transplantation either fetal pig thymus treated with 2DG and injected with human thymic stromal cells (represented by circles on the graphs), fetal pig thymus not treated with 2DG and injected with human thymic stromal cells (represented by squares on the graphs), and fetal pig thymus treated with 2DG and not injected with human thymic stromal cells (represented by triangles on the graph). FIG. 5A shows percentage of hCD45+ within white blood cells. FIG. 5B shows percentage of CD3+ cells in hCD45+. FIG. 5C shows hCD45+ cell count per μl of blood. FIG. 5D shows hCD3+ cell count per μl of blood. FIG. 5E shows percentage of CD4+ cells in hCD3+. FIG. 5F shows hCD4+ cell count per μl of blood. FIG. 5G shows percentage of CD19+ cells in hCD45+. FIG. 5H shows percentage of CD14+ cells in hCD45+. FIG. 5I shows hCD19+ cell count per μl of blood. FIG. 5J shows hCD14+ cell count per μl of blood. FIG. 5K shows the percentage of naïve cells in hCD3+. FIG. 5L shows percentage of effector memory cells in hCD3+.

FIG. 6 shows images of the detection of the injected human TECs admixed with pig TECs within the grafted thymus. Uninjected swine thymus grafted into a humanized mouse is shown on the left and a hybrid thymus grafted into a humanized mouse is shown on the right.

FIG. 7 show graphs of the in vitro T cell proliferation results which suggested that peripheral T cells of mice with hybrid thymus are partially tolerant of the human TEC donor. FIG. 7A shows the T cell proliferation response of mice with the various grafted thymi to pig dendritic cells. FIG. 7B shows the T cell proliferation response of mice with the various grafted thymi to human pig dendritic cells. Fetal pig thymus uninjected with human thymic stromal cells is represented by the circles, fetal pig thymus injected with human thymic stromal cells and not treated with 2DG are represented by squares, and fetal pig thymus injected with human thymic stromal cells treated with 2DG are represented by triangles.

FIG. 8 shows the increased responsiveness to human tissue restricted antigens (TRAs) (MART-1, NYESO1 and islet antigen IA-2) among human T cells that develop in a pig thymus (SW/HU mice) compared to those developing in a human thymus (HU/HU mice). FIG. 8A is a schematic of the mice models. FIG. 8B are graphs showing the proliferative responses of human peripheral T cells (18 weeks post-transplant) from the mice to human TRAs (IA-2, MART-1 and NYESO1) presented by human HSC donor DCs.

FIG. 9 shows that there is a lower survival of human Tregs and CD8 T cells that develop in a pig thymus (SW/HU mice) compared to those developing in a human thymus (HU/HU mice). FIG. 9 are graphs showing the various cells in the grafted thymic cells in HU/HU mice compared to SW/HU mice and of the various cells in the spleen/lymph node in HU/HU mice compared to SW/HU mice. HU/HU mice numbers are shown as circles, SW/HU mice numbers are shown as squares. FIG. 9A shows the total thymic cell count in the grafted thymi. FIG. 9B shows the percentage of thymic cell subsets in the grafted thymi. FIG. 9C shows the percentage of Tregs within SP-CD4+ in the grafted thymi. FIG. 9D shows the percental of Ki67+ cells in the grafted thymi. FIG. 9E shows the percentage of CD45RO+ cells in the grafted thymi. FIG. 9F shows the percentage of CTLA-4+ cells in the grafted thymi. FIG. 9G shows the total cell count in the spleen and lymph nodes (LN) in each subset of mice. FIG. 9H shows the percentage of hCD45+ cells in the spleen and lymph nodes (LN) in each subset of mice. FIG. 9I shows the percentage of T cells within hCD45+ in the spleen and lymph nodes (LN) in each subset of mice. FIG. 9J shows the percentage of CD4 and CD8 cells within T cells in the spleen and lymph nodes (LN) in each subset of mice. FIG. 9K shows the percentage of Tregs within CD4+ in the spleen and lymph nodes (LN) in each subset of mice. FIG. 9L shows the percentage of naïve cells in the spleen and lymph nodes (LN) in each subset of mice. FIG. 9M shows the percentage of EM cells in the spleen and lymph nodes (LN) in each subset of mice. FIG. 9N shows the percentage of HLA-DR+ cells the spleen and lymph nodes (LN) in each subset of mice. FIG. 9O shows the percentage of Ki67+ cells in the spleen and lymph nodes (LN) in each subset of mice. FIG. 9P shows the percentage of hCD45RO+ in the spleen and lymph nodes (LN) in each subset of mice. FIG. 9Q shows the percentage of CTLA-4+ in the spleen and lymph nodes (LN) in each subset of mice.

FIG. 10 shows the long-term (greater than 20 weeks) persistence of human TECs in “hybrid thymus”. FIG. 10A are images the grafted pig thymi including those not injected with human TECs (SW THY, top left panel), pig thymus injected with human fetal TECs (SW/Fetal hu-TES THY, top right panel), pig thymus injected with human pediatric TECs (SW/Pediatric hu-TEC THY, bottom left panel) and pig thymus injected with human hPSCs (SW/hPSC-TEC THY, bottom right panel). FIG. 10B are representative flow cytometric staining of gated CD45-negative cells in digested stroma from the various long-term thymic grafts showing the presence of EPCAM+, CD105-negative hu-TECs only in the human thymus (top right) and SW grafts that had been injected with hPSC-TEC progenitors (bottom left panel) but not in the uninjected SW THY grafts (top left). FIG. 10C is a graph of the quantification of the percentage of CD45− HLA-ABC+ EpCAM+ (injected human TECs) from multiple mice receiving human ES-TEC-injected SW thymus vs non-injected SW thymus.

FIG. 11 shows that the injection of hES-TECs into swine thymus promotes increased T cell development and increased peripheral CD4 and CD8 T cells. Swine thymus injected (green bar) or not (clear bar) with 1-2×10⁵ hES-TECs was grafted under the renal capsule of thymectomized NSG mice injected intravenously with 2×10⁵ human fetal liver-derived CD34⁺ cells. Splenocytes and thymocytes from thymic grafts were analyzed by flow cytometry 18-22 weeks post-transplant. FIG. 11A is a graph of absolute number of human CD3⁺ T cells in each group of mice. FIG. 11B is a graph of the absolute numbers of human CD8⁺ T cells in each group of mice. FIG. 11C is a graph of the absolute number of human CD4⁺ T cells in each group of mice. FIG. 11D is a graph of the absolute number of recent thymic emmigrant CD31⁺CD4⁺ naive cells defined as CD45RA⁺CCR7⁺ cells in mononuclear cells of the spleen in each group of mice. FIG. 11E are graphs of number of indicated cells in thymocytes in each group of mice stained for expression of HuCD45, CD19, CD14, CD4, CD8, CD45RA and CD45RO. Thymocytes were gated as huCD45⁺CD19⁻CD14⁻ cells. Absolute count of thymocytes from half the thymus graft gated as total human CD45 cells, double positive CD4⁺CD8⁺, single positive CD4⁺CD8 and CD4⁻CD8⁺ are shown. SP CD4 and CD8 cells were further subgated into immature CD45RO⁺ compared for more mature CD45RA⁺ thymocytes. Average+SEM are shown for swine thymus injected with hES-TEC (n=6, squares) and swine thymus alone (n=5, triangles) representative of two independent experiments. Thymic grafts yielding fewer than 6×10⁵ (n=1 each from SwTHY and SwTHY+TEC) cells were eliminated from analysis. Mann-Whitney test was used to determine p-values comparing SwTHY alone to SwTHY hES-TEC injected groups with p<0.05 considered significant. *p<0.05, ⁺p=0.05.

### DETAILED DESCRIPTION OF THE INVENTION

### Abbreviations

SW—swine
HU—human
TEC—thymic epithelial cells
TMC—thymic mesenchyme cells
WBC—white blood cells
DP—double positive cells (both CD4+, CD8+)
SP—single positive cells (either CD4+ or CD8+)
Tregs—regulatory T cells
LN—lymph nodes
TRA—tissue restricted antigens
2DG—2D glucose
MACS—magnetic activated cell sorting
HSCs—human hematopoietic cells

The present disclosure provides for a method to generate a human/pig hybrid thymus to achieve immune tolerance to pig antigens with optimal immune function of the human T-cell repertoire generated. The method overcomes the limitations encountered when using simple pig thymus by improving the function and self-tolerance of a human T cell in a pig thymus while allowing tolerance to the pig xenograft to develop.

The present method generates a hybrid pig-human thymus that contains patient-specific thymic epithelial cells (TECs). The patient-specific TECs may either be directly obtained from the patient's thymus, generated from patient-specific induced pluripotent stem cells, or generated from embryonic stem cells that naturally or artificially share human leukocyte antigen (HLA) alleles with the patient. The patient-specific TECs can participate in positive selection, resulting in T cells that more readily recognize foreign antigens presented by recipient HLA molecules in the periphery. Additionally, since many tissue-specific antigens (TSAs) differ between human and pig, the addition of human TECs to make human TSAs in the thymus helps assure protection from autoimmunity. As such, the use of a hybrid thymus instead of a pig thymus may improve the function and self-tolerance of the generated human T cell repertoire and enable induction of xenograft tolerance.

The present hybrid thymus/thymic tissue (e.g., a hybrid pig-human thymus/thymic tissue) may also be used for immune reconstitution in patients lacking adequate thymic function or having T-cell immunodeficiency (e.g. aging thymus in adults). Applications of the hybrid thymus/thymic tissue (e.g., a hybrid pig-human thymus/thymic tissue) include xenogeneic model for pharmacology and drug screening, medical xenogeneic transplant tolerance testing and preparation, and tolerance induction to therapeutic molecules that can cause immunogenicity (e.g. mAbs) used during transplant or immune disorders treatment.

As used herein, a hybrid thymic tissue refers to a thymic tissue from the donor mammal of a second species and comprises thymic epithelial cells from the recipient mammal of a first species.

In one embodiment, a hybrid thymus/thymic tissue is constructed where a pig thymus/thymic tissue (e.g., a fetal thymus/thymic tissue) containing human (e.g., patient-specific) thymic epithelial cells (TECs) obtained from a human's thymus/thymic tissue (e.g., the patient's thymus/thymic tissue). In another embodiment, a hybrid thymus/thymic tissue is constructed where a pig thymus/thymic tissue (e.g., a fetal thymus/thymic tissue) containing human (e.g., patient-specific) TECs generated from human (e.g., patient-specific) induced pluripotent stem cells. In yet another embodiment, a hybrid thymus/thymic tissue is constructed where a pig thymus/thymic tissue (e.g., a fetal thymus/thymic tissue) containing human TECs generated from embryonic stem cells that either naturally share HLA alleles with the patient or have been engineered to do so.

Inclusion of human TECs to the pig thymus graft can have functional effects on antigen recognition.

The present disclosure provides for a method of inducing tolerance in a recipient mammal of a first species to a graft obtained from a donor mammal of a second species. The method may contain the following steps: (a) introducing a hybrid thymic tissue into the recipient mammal, wherein the hybrid thymic tissue is a thymic tissue from the second species and comprises thymic epithelial cells from the first species; and (b) implanting the graft from the donor mammal in the recipient mammal. The donor may be swine including a miniature swine. The recipient may be human.

The present disclosure also provides for a method to generate a primate-pig hybrid thymus/thymic tissue to achieve immune tolerance to pig antigens with optimal immune function of the primate T-cell repertoire generated. One embodiment of the present disclosure provides for a hybrid thymus/thymic tissue in the pig to baboon.

The hybrid thymus/thymic tissue may be implanted as a primarily vascularized thymus lobe or composite thymo-kidney graft.

The hybrid thymus/thymic tissue may be transplanted intramuscularly in the recipient. The hybrid thymus/thymic tissue may be transplanted either into the quadriceps muscle alone or with additional transplantation sites (e.g., kidney capsule and omentum) in the recipient. Wu et al., Xenogeneic Thymus Transplantation in A Pig-to-baboon Model, Transplantation, 2003, 75(3): 282-291.

The recipient of the xenotransplantation is a mammal of a first mammalian species. The donor of the xenotransplantation refers to a mammal of a second mammalian species. The donor mammal is the donor of cells, tissues, and/or organs for the xenotransplantation.

The present disclosure provides for a method of inducing tolerance in a recipient mammal of a first species to a graft obtained from a donor mammal of a second species, the method comprising the steps of: (a) introducing a hybrid thymic tissue into the recipient mammal, wherein the hybrid thymic tissue is a thymic tissue from the second species and comprises thymic epithelial cells from the first species; and (b) implanting the graft from the donor mammal in the recipient mammal. Step (a) may be conducted prior to, or simultaneous with, step (b).

The present disclosure also provides for a method of restoring or inducing immunocompetence in a recipient mammal of a first species, the method comprising the step of introducing a hybrid thymic tissue into the recipient mammal, wherein the hybrid thymic tissue is a thymic tissue from a donor mammal of a second species and comprises thymic epithelial cells from the first species.

Also encompassed by the present disclosure is a method of restoring or promoting thymus-dependent ability for T cell progenitors to develop into mature functional T cells in a recipient mammal of a first species, the method comprising introducing a hybrid thymic tissue into the recipient mammal, wherein the hybrid thymic tissue is a thymic tissue from a donor mammal of a second species and comprises thymic epithelial cells from the first species.

In one embodiment, thymic function is essentially absent in the recipient mammal before a hybrid thymic tissue is introduced. In another embodiment, the recipient mammal is thymectomized before a hybrid thymic tissue is introduced. In yet another embodiment, the recipient mammal has an immune disorder.

The second species may be swine, such as a miniature swine.

The first species is may be primate, such as non-human primate or human.

In one embodiment, the recipient mammal is a human and the donor mammal is a miniature swine.

The thymic tissue from the second species may be a fetal thymic tissue, or a neonatal thymic tissue.

The thymic epithelial cells from the first species may be obtained from the recipient mammal's thymus. The thymic epithelial cells from the first species may be generated from the recipient mammal's induced pluripotent stem cells (iPSCs). The thymic epithelial cells from the first species may be generated from embryonic stem cells that share HLA alleles with the recipient mammal. For example, the embryonic stem cells may naturally share HLA alleles with the recipient mammal or are genetically engineered to share HLA alleles with the recipient mammal.

In one embodiment, the hybrid thymic tissue is implanted in the recipient mammal. For example, the hybrid thymic tissue may be implanted as a primarily vascularized thymus lobe or composite thymo-kidney graft.

The hybrid thymic tissue may be generated by introducing thymic epithelial cells from the first species into the thymic tissue from the second species. The hybrid thymic tissue may be generated by injecting thymic epithelial cells from the first species into the thymic tissue from the second species.

The hybrid thymic tissue may be generated by a method comprising the following steps: (i) treating the thymic tissue from the second species with 2-deoxyglucose (2DG); and (ii) introducing thymic epithelial cells from the first species into the 2DG-treated thymic tissue. In step (ii) the thymic epithelial cells may be suspended in biomaterial, such as Matrigel, before being injected to the 2DG-treated thymic tissue.

The thymic epithelial cells may be suspended in a biomaterial (e.g., Matrigel) before being injected to the thymic tissue from the second species.

In some embodiments, before being introduced into the thymic tissue from the second species, the thymic epithelial cells from the first species may be combined with (e.g., be suspended in) a biomaterial. The biomaterial may be a sol-gel, a hydrogel laden with proteins, a Matrigel, an artificially constructed scaffold with cells, and combinations thereof. Non-limiting examples of the biomaterials may also include, polyethylene-imine and dextran sulfate, poly(vinylsiloxane)ecopolymerepolyethyleneimine, phosphorylcholine, poly(ethylene glycol), poly(lactic-glycolic acid), poly(lactic acid), polyhydroxyvalerte and copolymers, polyhydroxybutyrate and copolymers, polydiaxanone, polyanhydrides, poly(amino acids), poly(orthoesters), polyesters, collagen, gelatin, cellulose polymers, chitosans, alginates, fibronectin, extracellular matrix proteins, vinculin, agar, agarose, hyaluronic acid, matrigel and combinations thereof.

The present method may further comprise administering hematopoietic stem cells (HSCs) to the recipient mammal.

The graft may comprise cells, a tissue or an organ. In one embodiment, the graft comprises hematopoietic stem cells. In another embodiment, the graft comprises bone marrow. In yet another embodiment, the graft comprises a heart, a kidney, a liver, a pancreas, a lung, an intestine, skin, a small bowel, a trachea, a cornea, or combinations thereof.

The present disclosure provides for an isolated hybrid thymic tissue comprising thymic epithelial cells from a first mammalian species and a thymic tissue from a second mammalian species.

An alternative approach has been developed to achieve central T cell tolerance of highly disparate xenogeneic donors that involve transplantation of a porcine thymus to an immunocompetent, T cell-depleted and thymectomized recipient. These studies were initiated in mice, which demonstrated marked and specific unresponsiveness in vitro and prolongation of donor-specific skin graft survival. Lee L A, Gritsch H A, Sergio J J, et al. Specific tolerance across a discordant xenogeneic transplantation barrier. Proc Natl Acad Sci USA. 1994; 91:10864-10867. Zhao Y, Swenson K, Sergio J J, Arn J S, Sachs D H, Sykes M. Skin graft tolerance across a discordant xenogeneic barrier. Nature Med. 1996; 2:1211-1216. The murine model permitted extensive studies of the mechanisms of tolerance and of immune function conferred by T cell reconstitution in a xenogeneic thymic graft. Intrathymic clonal deletion is a major mechanism tolerizing newly-developing thymocytes to the xenogeneic donor and the recipient. Zhao Y, Sergio J J, Swenson K A, Am J S, Sachs D H, Sykes M. Positive and negative selection of functional mouse CD4 cells by porcine MHC in pig thymus grafts. J Immunol. 1997; 159:2100-2107. Zhao Y, Rodriguez-Barbosa J I, Shimizu A, Swenson K, Sachs D H, Sykes M. Despite efficient intrathymic negative selection of host-reactive T cells, autoimmune disease may develop in porcine thymus-grafted athymic mice: Evidence for failure of regulatory mechanisms suppressing autoimmunity. Transplantation. 2002; 75:1832-1840. Additional studies implicated Tregs developing in the porcine thymus graft in the suppression of residual mouse anti-pig responses. Zhao et al. The induction of specific pig skin graft tolerance by grafting with neonatal pig thymus in thymectomized mice. Transplantation. 2000; 69:1447-1451. Rodriguez-Barbosa et al. Enhanced CD4 reconstitution by grafting neonatal porcine tissue in alternative locations is associated with donor-specific tolerance and suppression of pre-existing xenoreactive T cells. Transplantation. 2001; 72:1223-1231. Using T cell receptor (TCR) transgenic recipient mice of different MHC haplotypes and TCRs for which positively and negatively selecting murine MHC alleles had been identified previously, it was demonstrated that positive selection in a porcine thymus graft was mediated exclusively by the porcine thymic MHC, with no contribution from the murine hematopoietic cells, whereas negative selection was mediated by both the pig and the mouse MHC, consistent with the presence of class II MHC+ APCs from both species in the donor pig thymus grafts. Zhao Y, Rodriguez-Barbosa J I, Zhao G, Shaffer J, Am J S, Sykes M. Maturation and function of mouse T cells with a transgeneic TCR positively selected by highly disparate xenogeneic porcine MHC. Cell Mol Biol. 2000; 47:217-228. Zhao Y, Swenson K, Sergio J J, Sykes M. Pig MHC mediates positive selection of mouse CD4+ T cells with a mouse MHC-restricted TCR in pig thymus grafts. J Immunol. 1998; 161:1320-1326. Remarkably, despite the lack of murine MHC participation in positive selection and the complete MHC disparity of the porcine thymus and the murine recipient, these T cells were able to respond to immunization with protein antigens presented by murine MHC molecules and, most importantly to protect the mice from an opportunistic pathogen whose clearance was dependent on CD4+ T cells. Zhao et al. Immune restoration by fetal pig thymus grafts in T cell-depleted, thymectomized mice. J Immunol. 1997; 158:1641-1649. These results are interpreted as demonstrating that sufficient cross-reactivity for recognition of foreign antigens on recipient MHC can occur if a diverse T cell repertoire is selected in a xenogeneic thymic graft.

The porcine thymic transplantation approach to tolerance has been extended to the humanized mouse model to provide proof-of-principle that human T cells can develop normally and are centrally tolerized to porcine xenoantigens in pig thymic grafts. Nikolic B, Gardner J P, Scadden D T, Am J S, Sachs D H, Sykes M. Normal development in porcine thymus grafts and specific tolerance of human T cells to porcine donor MHC. J Immunol. 1999; 162:3402-3407. Kalscheuer H O, T.; Dahmani, A.; Li, H.; Holzl, M.; Yamada, K.; Sykes, M. Xenograft tolerance and immune function of human T cell developing in pig thymus xenografts. J Immunology. 2014; 192(7):3442-3450. Both thymic and peripheral human T cells developing in a porcine thymus graft show specific unresponsiveness to the donor pig, with intact responses to third party pigs and allogeneic humans in mixed lymphocyte reactions (MLRs). These T cells also show unresponsiveness to the human hematopoietic stem cell (HSC) donor and the murine recipient in MLRs, reflecting the contribution of human donor APCs and murine APCs, both of which are detected in thymic xenografts, to negative selection. Kalscheuer et al. A model for personalized in vivo analysis of human immune responsiveness. Science Translational Medicine. 2012; 4(125):125ra130. Importantly, donor-specific skin graft tolerance is observed for human T cells developing in a porcine thymus graft.

Based on results in the murine model, the thymic xenotransplantation approach to tolerance has been extended to the large animal pig-to-baboon species combination. Initial studies using porcine thymic fragments placed under the kidney capsule of the baboon demonstrated some T cell recovery, donor-specific hyporesponsiveness in vitro and prolongation of donor skin graft survival compared to controls. However, the amount of pig thymic tissue that was implanted and vascularized was quite limited. Wu et al. Xenogeneic thymus transplantation in a pig-to-baboon model. Transplantation. 2003; 75(3):282-291. In order to achieve more robust thymic function and, in view of the murine data cited above, expecting that donor-specific Tregs developing in a pig thymus would be needed to suppress pre-existing T cells not depleted by the conditioning regimen, subsequent studies utilized a primarily vascularized pig thymus, which had already shown efficacy in tolerance induction in an allogeneic pig kidney transplant model. Yamada K, Shimizu A, Utsugi R, et al. Thymic transplantation in miniature swine. II. Induction of tolerance by transplantation of composite thymokidneys to thymectomized recipients. J Immunol. 2000; 164:3079-3086. Thymi were transplanted either as part of a composite “thymokidney” graft prepared in the donor pig several months earlier by placing autologous thymic fragments under the pig's kidney capsule or by direct vascular anastomosis of a pig thymic lobe in a baboon. Yamada K, Yazawa K, Shimizu A, et al. Marked prolongation of porcine renal xenograft survival in baboons through the use of alpha1,3-galactosyltransferase gene-knockout donors and the cotransplantation of vascularized thymic tissue. Nature medicine. 2005; 11(1):32-34. Both approaches led, for the first time, to long-term survival of GalT knockout pig kidneys in baboons. Tasaki et al., Rituximab treatment prevents the early development of proteinuria following pig-to-baboon xeno-kidney transplantation. Journal of the American Society of Nephrology: JASN. 2014; 25(4):737-744. Survival of animals receiving this treatment has been limited by thrombotic complications of anti-CD40L and by proteinuria due to a minimal change disease-like glomerulopathy, which can be avoided by using non-thrombogenic anti-CD40 and by administering rituximab and CTLA4Ig, respectively. Yamada et al., Xenotransplantation: Where Are We with Potential Kidney Recipients? Recent Progress and Potential Future Clinical Trials. Curr Transplant Rep. 2017; 4(2):101-109.

Baboons receiving porcine thymokidney grafts have shown evidence of de novo recipient (baboon) thymopoiesis in the porcine thymic graft, appearance of recent thymic emigrants in the periphery and donor-specific unresponsiveness in Elispot and MLR assays, as well as a decline in non-Gal natural antibodies. Tanabe et al. Role of Intrinsic (Graft) Versus Extrinsic (Host) Factors in the Growth of Transplanted Organs Following Allogeneic and Xenogeneic Transplantation, Am J Transplant. 2017 July; 17(7):1778-1790. While the latter may reflect absorption by the pig kidney, minimal IgM binding was detected on these xenografts, with no complement fixation or significant pathology. Thus, the results obtained with this model demonstrate the potential of composite thymus-kidney xenografts to induce tolerance in primates.

Limitations of generating a human T cell repertoire in a xenogeneic porcine thymus include the preferential recognition of microbial antigens on porcine MHC, which would be useful for protecting the graft but would not optimize protection against microbial pathogens infecting the host, as well as the failure to negatively select conventional T cells and positively select Tregs recognizing human tissue-restricted antigens (TRAs). Indeed, studies in humanized mice have shown reduced responses to peptides presented by human APCs following immunization when the human T cells developed in a pig rather than a human thymus graft.

The approach to overcome this limitation involves creation of a “hybrid thymus”, in which recipient thymic epithelial cells obtained either from thymectomy specimens or generated from stem cells are injected into the porcine thymic tissue. Hybrid thymi from post-natal thymus donors have been generated, where the hybrid thymus promotes tolerance to human TRAs among human T cells.

Pig thymus grafts have been shown to support the development of normal, diverse murine or human T cell repertoires and that these T cells are specifically tolerant of the xenogeneic pig donor. However, recognition of foreign antigens presented by recipient HLA molecules in the periphery is suboptimal. Thus, immune function may be less than optimal. As shown herein, this can be overcome by providing recipient TECs in the pig-human hybrid thymus graft because these TECs will participate in positive selection, resulting in T cells that can more readily recognize foreign antigens presented by recipient HLA molecules in the periphery. For pig thymus grafts, survival, homeostasis and function of T cells that do not find their “positive selecting” ligand in the periphery is suboptimal. The positive selecting ligand is the MHC/peptide complex on TECs that rescue thymocytes from programmed cell death when the thymocyte has a low affinity T cell receptor recognizing that complex. Providing recipient TECs in the pig-human hybrid thymus allows positive selection of T cells that will find the same ligand on recipient cells in the periphery, conferring normal survival, homeostasis and function. For pig thymus grafts, TECs produce antigens that are otherwise expressed only in very specific tissues in the periphery (i.e., tissue-specific antigens, TSAs). Two important consequences of this expression of TSAs by TECs are: a) clonal deletion of thymocytes that strongly recognize them, removing these autoreactive T cells from the repertoire; b) positive selection of regulatory T cells recognizing them, adding a safety net to prevent autoimmunity in the periphery. Since many TSAs differ between human and pig, the addition of human TECs to make human TSAs in the pig-human hybrid thymus graft will help to assure protection from autoimmunity.

In summary, the use of a hybrid thymus instead of a simple pig thymus can improve the function and self-tolerance of a human T cell repertoire generated in a pig thymus while allowing tolerance to the pig to develop.

In one embodiment, the recipient is thymectomized. In another embodiment, the recipient is not thymectomized. In yet another embodiment, the recipient has a low rate of thymopoiesis due to age. In still another embodiment, the recipient has a senescent thymus.

Thymic xenotransplantation using the present hybrid thymus/thymic tissue may or may not be combined with mixed chimerism induction. For example, when thymic xenotransplantation is combined with mixed chimerism induction with durable mixed pig-human chimerism, both pig and human APCs would be present in the native human thymus and the porcine thymic xenograft, ensuring lifelong negative selection of thymocytes recognizing either pig or human antigens expressed on hematopoietic cells. Moreover, conventional T cells recognizing pig or human TRAs would be deleted in the relevant species' thymus and those escaping deletion due to development in the thymus of the opposite species would be adequately suppressed by TRA-specific Tregs developing in the other thymus. The mixed porcine chimerism would assure tolerance of natural antibodies recognizing unknown xenogeneic targets and NK cells would be tolerized as well.

Xenotransplantation lends itself to tolerance induction more readily than allotransplantation from deceased human donors, as the ability to perform xenotransplantation electively permits the application of a tolerance protocol (e.g. mixed chimerism induction) in advance of the organ xenograft. In one embodiment, the present method involves tolerizing the immune system of the recipient first, confirming that tolerance has been achieved and subsequently performing the organ transplant without immunosuppression or with a shortened course of immunosuppression.

The present disclosure provides a method of inducing tolerance in a recipient mammal of a first species (e.g., a primate such as a human) to a graft obtained from a mammal of a second species, e.g., a swine. The method includes: prior to or simultaneous with transplantation of the graft, introducing into the recipient mammal a hybrid thymus/thymic tissue; and (optionally) implanting the graft in the recipient. The hybrid thymus/thymic tissue prepares the recipient for the graft that follows, by inducing immunological tolerance at the T-cell level.

The present disclosure provides methods for inducing xenograft tolerance in a recipient, the methods including the step of introducing a hybrid thymus/thymic tissue into the recipient.

In one embodiment, host T cells of an athymic, T cell-depleted recipient which has received a hybrid thymus/thymic tissue, can mature in the hybrid thymus/thymic tissue. Host T cells which mature in the implanted hybrid thymus/thymic tissue are immunocompetent.

The present disclosure provides a method of restoring or inducing immunocompetence (or restoring or promoting the thymus-dependent ability for T cell progenitors to mature or develop into functional mature T cells) in a host or recipient, e.g., a primate host or recipient, e.g., a human, which is capable of producing T cell progenitors but which is thymus-function deficient and thus unable to produce a sufficient number of mature functional T cells for a normal immune response. The method includes the step of introducing into the recipient, a hybrid thymus/thymic tissue, so that host T cells can mature in the implanted hybrid thymus/thymic tissue.

In one embodiment, the recipient/host is a primate, e.g., a human, and the donor is swine, e.g., miniature swine.

The method can include other steps which facilitate acceptance of the hybrid thymus/thymic tissue, or otherwise optimize the method. In certain embodiments, liver or spleen tissue, such as fetal or neonatal liver or spleen tissue, is implanted with the thymic tissue; donor hematopoietic cells, e.g., cord blood stem cells or fetal or neonatal liver or spleen cells, are administered to the recipient, e.g., a suspension of fetal liver cells is administered intraperitoneally or intravenously. The recipient may be thymectomized, such as before or at the time the hybrid thymus/thymic tissue is introduced.

In certain embodiments, the method includes: (preferably prior to or at the time of introducing the thymic tissue into the recipient) depleting, inactivating or inhibiting recipient natural killer (NK) cells, e.g., by introducing into the recipient an antibody capable of binding to NK cells of the recipient, to prevent NK mediated rejection of the thymic tissue; (preferably prior to or at the time of introducing the thymic tissue into the recipient) depleting, inactivating or inhibiting host T cell function, e.g., by introducing into the recipient an antibody capable of binding to T cells of the recipient; (preferably prior to or at the time of introducing the thymic tissue into the recipient) depleting, inactivating or inhibiting host CD4+ cell function, e.g., by introducing into the recipient an antibody capable of binding to CD4, or CD4+ cells of the recipient.

Certain embodiments include the step of (preferably prior to thymic tissue or hematopoietic stem cell transplantation) creating hematopoietic space, e.g., by one or more of: irradiating the recipient mammal with low dose, e.g., between about 100 and 400 rads, whole body irradiation, the administration of a myelosuppressive drug, or the administration of a hematopoietic stem cell inactivating or depleting antibody, to deplete or partially deplete the bone marrow of the recipient (preferably prior to thymic tissue transplantation).

Certain embodiments include (preferably prior to thymic tissue or hematopoietic stem cell transplantation) inactivating thymic T cells by one or more of: irradiating the host with, e.g., about 700 rads of thymic irradiation, administering to the recipient one or more doses of an anti T cell antibody, e.g., an anti-CD4 and/or an anti-CD8 monoclonal antibody, or administering to the recipient a short course of an immunosuppressant.

Certain embodiments include depleting or otherwise inactivating natural antibodies, e.g., by one or more of: the administration of a drug which depletes or inactivates natural antibodies, e.g., deoxyspergualin; the administration of an anti-IgM antibody, or the adsorption of natural antibodies from the host's blood, e.g., by contacting the host's blood with donor antigen, e.g., by hemoperfusion of a donor organ, e.g., a kidney or a liver, from the donor species.

Other methods may be combined with the methods disclosed herein to promote the acceptance of the graft by the recipient. For example, tolerance to the thymic tissue can also be induced by inserting a nucleic acid which expresses a donor antigen, e.g., a donor MHC gene, into a cell of the recipient, e.g., a hematopoietic stem cell, and introducing the genetically engineered cell into the recipient. For example, human recipient stem cells can be engineered to express a swine MHC gene, e.g., a swine class I or class II MHC gene, or both a class I and class II gene, and the cells implanted in a human recipient who will receive the hybrid thymic tissue. When inserted into a recipient primate, e.g., a human, expression of the donor MHC gene results in tolerance to subsequent exposure to donor antigen, and can thus induce tolerance to the thymic tissue.

Methods of inducing tolerance, e.g., by the implantation of hematopoietic stem cells, can also be combined with the methods disclosed herein.

Other methods of inducing tolerance may also be used to promote acceptance of the thymic tissue. For example, suppression of helper T cells, which can be induced, e.g., by the administration of a short course of high dose immunosuppressant, e.g., cyclosporine, has been found to induce tolerance. In these methods, helper T cells is suppressed for a comparatively short period just subsequent to implantation of a graft, and does not require or include chronic immunosuppression.

Other methods of promoting tolerance or promoting the acceptance of donor tissue, e.g., by altering levels of cytokine activity, or inhibiting Graft-versus-recipient-disease, may also be used to in combination with the present methods.

In another aspect, the present disclosure provides a method of diminishing or inhibiting T cell activity, preferably the activity of thymic or lymph node T cells, in a recipient mammal, e.g., a primate, e.g., a human, which receives a graft from a donor mammal. The method includes, inducing tolerance to the graft; administering to the recipient a short course of an immunosuppressive agent, e.g., cyclosporine, sufficient to inactivate T cells, preferably thymic or lymph node T cells.

“Thymus-function deficient”, as used herein, refers to a condition in which the ability of an individual's thymus to support the maturation of T cells is impaired as compared with a normal individual. Thymus deficient conditions include those in which the thymus or thymus function is essentially absent.

“Tolerance”, as used herein, refers to the inhibition or decrease of a graft recipient's ability to mount an immune response, e.g., to a donor antigen, which would otherwise occur, e.g., in response to the introduction of a non self MHC antigen into the recipient. Tolerance can involve humoral, cellular, or both humoral and cellular responses. The concept of tolerance includes both complete and partial tolerance. In other words, as used herein, tolerance include any degree of inhibition of a graft recipient's ability to mount an immune response, e.g., to a donor antigen.

“Hematopoietic stem cell”, as used herein, refers to a cell that is capable of developing into mature myeloid and/or lymphoid cells. Preferably, a hematopoietic stem cell is capable of the long-term repopulation of the myeloid and/or lymphoid lineages. Stem cells derived from the cord blood of the recipient or the donor can be used in methods of the disclosure.

“Miniature swine”, as used herein, refers to completely or partially inbred miniature swine.

“Graft”, as used herein, refers to a body part, organ, tissue, cells, or portions thereof.

“Stromal tissue”, as used herein, refers to the supporting tissue or matrix of an organ, as distinguished from its functional elements or parenchyma.

Restoring, inducing, or promoting immunocompetence, as used herein, means one or both of: (1) increasing the number of mature functional T cells in the recipient (over what would be seen in the absence of treatment with a method of the disclosure) by either or both, increasing the number of recipient-mature functional T cells or by providing mature functional donor-T cells, which have matured in the recipient; or (2) improving the immune-responsiveness of the recipient, e.g., as is measured by the ability to mount a skin response to a recall antigen, or improving the responsiveness of a T cell of the recipient, e.g., as measured by an in vitro test, e.g., by the improvement of a proliferative response to an antigen, e.g., the response to tetanus antigen or to an alloantigen.

Restoring or inducing the thymus-dependent ability for T cell progenitors to mature into mature T cells, as used herein, means either or both, increasing the number of functional mature T cells of recipient origin in a recipient, or providing mature functional donor T cells to a recipient, by providing donor thymic tissue in which T cells can mature. The increase can be partial, e.g., an increase which does not bring the level of mature functional T cells up to a level which results in an essentially normal immune response or partial, e.g., an increase which falls short of bringing the recipient's level of mature functional T cells up to a level which results in an essentially normal immune response.

In certain embodiments, preparation of the recipient for either organ transplantation or thymus replacement includes any or all of the following steps. They may be carried out in the following sequence.

First, a preparation of horse anti-human thymocyte globulin (ATG) is intravenously injected into the recipient. The antibody preparation eliminates mature T cells and natural killer cells. If not eliminated, mature T cells might promote rejection of both the thymic transplant and, after sensitization, the xenograft organ. The ATG preparation also eliminates natural killer (NK) cells. NK cells probably have no effect on an implanted organ, but might act immediately to reject the newly introduced thymic tissue. Anti-human ATG obtained from any mammalian host can also be used, e.g., ATG produced in pigs, although thus far preparations of pig ATG have been of lower titer than horse-derived ATG. ATG is superior to anti-NK monoclonal antibodies, as the latter are generally not lytic to all host NK cells, while the polyclonal mixture in ATG is capable of lysing all host NK cells. Anti-NK monoclonal antibodies can, however, be used. In a relatively severely immunocompromised individual this step may not be necessary. As host (or donor) T cells mature in the xenogeneic thymus they will be tolerant of the thymic tissue. Alternatively, as the host immune system is progressively restored, it may be desirable to treat the host to induce tolerance to the thymic tissue.

Optimally, the recipient can be thymectomized. In thymectomized recipients, recipient T cells do not have an opportunity to differentiate in the recipient thymus, but must differentiate in the hybrid thymic tissue. In some cases, it may be necessary to splenectomize the recipient in order to avoid anemia.

Second, the recipient can be administered low dose radiation. Although this step is thought to be beneficial in bone marrow transplantation (by creating hematopoietic space for newly injected bone marrow cells), it is of less importance in thymic grafts which are not accompanied by bone marrow transplantation. However, a sublethal dose e.g., a dose about equal to 100, or more than 100 and less than about 400, rads, whole body radiation, plus 700 rads of local thymic radiation, can be used.

Third, natural antibodies can be adsorbed from the recipient's blood. Antibody removal can be accomplished by exposing the recipient's blood to donor or donor species antigens, e.g., by hemoperfusion of a liver of the donor species to adsorb recipient-natural antibodies. Pre-formed natural antibodies (nAb) are the primary agents of graft rejection. Natural antibodies bind to xenogeneic endothelial cells and are primarily of the IgM class. These antibodies are independent of any known previous exposure to antigens of the xenogeneic donor. B cells that produce these natural antibodies tend to be T cell-independent, and are normally tolerized to self antigen by exposure to these antigens during development. Again, this step may not be required, at least initially, in a relatively severely immunocompromised patient.

The hybrid thymic tissue is implanted in the recipient. Fetal or neonatal liver or spleen tissue can be included.

One, or any combination including all, of these procedures may aid the survival of implanted thymic tissue or another xenogeneic organ.

Methods of the present disclosure can be used to confer tolerance to xenogeneic grafts, e.g., wherein the graft donor is a nonhuman animal, e.g., a swine, e.g., a miniature swine, and the graft recipient is a primate, e.g., a human.

The donor of the xenograft and the individual that supplies the tolerance-inducing thymic tissue may be the same individual or may be as closely related as possible. For example, it is preferable to derive a xenograft from a colony of donors that is highly or completely inbred.

The second mammalian species (i.e., the donor) may be a non-human mammalian species, such as a swine species (e.g., a miniature swine species) or a non-human primate species. Non-limiting examples of the first mammalian species include a swine, rodent, non-human primate, cow, goat, and horse.

In one embodiment, the second mammalian species (i.e., the donor) is a miniature swine which is at least partially inbred (e.g., the swine is homozygous at swine leukocyte antigen (SLA) loci, and/or is homozygous at at least 65%, 70%, 75%, 80%, 85%, 90%, 95%, or more, of all other genetic loci). The genetic engineering can be made in wholly or partially inbred swine (e.g., miniature swine, transgenic swine, etc.). For example, inbred Massachusetts General Hospital (MGH) miniature swine may be used in the present methods. These include the MGH miniature swine which have been inbred for over 40 years and are homozygous at all genetic loci. In one embodiment, inbred SLA^{dd} miniature swine may be used. Mezrich et al. and Sachs, Histocompatible miniature swine: An inbred large-animal model. Transplantation, 2003; 75:904-907. Swine at National Swine Resource and Research Center (NSRRC, RADIL, University Missouri, Columbia Mo.) may also be used in the present methods.

The first mammalian species (i.e., the recipient) may be a primate, such as non-human primate (e.g., a baboon, or cynomolgus monkey) or human. In one embodiment, the second species is human.

In various embodiments, the donor (second species) and recipient (first species) are of different species. For example, the donor is a non-human animal, e.g., a miniature swine, and the recipient is a human.

Also encompassed by the present disclosure are methods of transplanting a graft from such a donor animal of the second mammalian species into a recipient mammal of a first mammalian species (e.g., human).

Cells, tissues, organs or body fluids of the present transgenic donor animal may be used for transplantation (e.g., xenotransplantation). The graft harvested from the donor animal for transplantation may include, but are not limited to, a heart, a kidney, a liver, a pancreas, a lung transplant, an intestine, skin, thyroid, bone marrow, small bowel, a trachea, a cornea, a limb, a bone, an endocrine gland, blood vessels, connective tissue, progenitor stem cells, blood cells, hematopoietic cells, Islets of Langerhans, brain cells and cells from endocrine and other organs, bodily fluids, and combinations thereof.

The cell can be any type of cell. In certain embodiments, the cell is a hematopoietic cell (e.g., a hematopoietic stem cell, lymphocyte, a myeloid cell), a pancreatic cell (e.g., a beta-islet cell), a kidney cell, a heart cell, or a liver cell.

Bone marrow cells (BMC), or hematopoietic stem cells (e.g., a fetal liver suspension or mobilized peripheral blood stem cells) of the donor animal may be injected into the recipient.

The method may also include one or more of the following treatments: a treatment which inhibits T cells, blocks complement, or otherwise down-regulates the recipient immune response to the graft.

Treatments that promote tolerance and/or decrease immune recognition of the graft include use of immunosuppressive agents (e.g., cyclosporine, FK506), antibodies (e.g., anti-T cell antibodies such as polyclonal anti-thymocyte antisera (ATG), and/or a monoclonal anti-human T cell antibody, such as LoCD2b), irradiation, and methods to induce mixed chimerism. U.S. Pat. Nos. 6,911,220; 6,306,651; 6,412,492; 6,514,513; 6,558,663; and U.S. Pat. No. 6,296,846. Kuwaki et al., Nature Med., 11(1):29-31, 2005. Yamada et al., Nature Med. 11 (1):32-34, 2005.

In some embodiments, the recipient is thymectomized and/or splenectomized. Thymic irradiation can be used.

In some embodiments, the recipient is administered low dose radiation (e.g., a sublethal dose of between 100 rads and 400 rads whole body radiation). Local thymic radiation may also be used.

The recipient can be treated with an agent that depletes complement, such as cobra venom factor.

Natural antibodies can be eliminated by organ perfusion, and/or transplantation of tolerance-inducing bone marrow. Natural antibodies can be absorbed from the recipient's blood by hemoperfusion of a liver of the donor species. The cells, tissues, or organs used for transplantation may be genetically modified such that they are not recognized by natural antibodies of the host (e.g., the cells are a-1,3-galactosyltransferase deficient).

In some embodiments, the methods include treatment with a human anti-human CD154 mAb, mycophenolate mofetil, and/or methylprednisolone. The methods can also include agents useful for supportive therapy such as anti-inflammatory agents (e.g., prostacyclin, dopamine, ganiclovir, levofloxacin, cimetidine, heparin, antithrombin, erythropoietin, and aspirin).

In some embodiments, donor stromal tissue is administered.

An immunosuppressant, also referred to as an immunosuppressive agent, can be any compound that decreases the function or activity of one or more aspects of the immune system, such as a component of the humoral or cellular immune system or the complement system.

Non-limiting examples of immunosuppressants include, (1) antimetabolites, such as purine synthesis inhibitors (such as inosine monophosphate dehydrogenase (IMPDH) inhibitors, e.g., azathioprine, mycophenolate, and mycophenolate mofetil), pyrimidine synthesis inhibitors (e.g., leflunomide and teriflunomide), and antifolates (e.g., methotrexate); (2) calcineurin inhibitors, such as tacrolimus, cyclosporine A, pimecrolimus, and voclosporin; (3) TNF-alpha inhibitors, such as thalidomide and lenalidomide; (4) IL-1 receptor antagonists, such as anakinra; (5) mammalian target of rapamycin (mTOR) inhibitors, such as rapamycin (sirolimus), deforolimus, everolimus, temsirolimus, zotarolimus, and biolimus A9; (6) corticosteroids, such as prednisone; and (7) antibodies to any one of a number of cellular or serum targets (including anti-lymphocyte globulin and anti-thymocyte globulin).

Non-limiting exemplary cellular targets and their respective inhibitor compounds include, but are not limited to, complement component 5 (e.g., eculizumab); tumor necrosis factors (TNFs) (e.g., infliximab, adalimumab, certolizumab pegol, afelimomab and golimumab); IL-5 (e.g., mepolizumab); IgE (e.g., omalizumab); BAYX (e.g., nerelimomab); interferon (e.g., faralimomab); IL-6 (e.g., elsilimomab); IL-12 and IL-13 (e.g., lebrikizumab and ustekinumab); CD3 (e.g., muromonab-CD3, otelixizumab, teplizumab, visilizumab); CD4 (e.g., clenoliximab, keliximab and zanolimumab); CD11a (e.g., efalizumab); CD18 (e.g., erlizumab); CD20 (e.g., afutuzumab, ocrelizumab, pascolizumab); CD23 (e.g., lumiliximab); CD40 (e.g., teneliximab, toralizumab); CD62L/L-selectin (e.g., aselizumab); CD80 (e.g., galiximab); CD147/basigin (e.g., gavilimomab); CD154 (e.g., ruplizumab); BLyS (e.g., belimumab); CTLA-4 (e.g., ipilimumab, tremelimumab); CAT (e.g., bertilimumab, lerdelimumab, metelimumab); integrin (e.g., natalizumab); IL-6 receptor (e.g., tocilizumab); LFA-1 (e.g., odulimomab); and IL-2 receptor/CD25 (e.g., basiliximab, daclizumab, inolimomab).

Natural antibodies of the recipient may be eliminated by organ perfusion, and/or transplantation of tolerance-inducing bone marrow.

In one embodiment, a recipient is treated with a preparation of horse anti-human thymocyte globulin (ATG) injected intravenously (e.g., at a dose of approx. 25-100 mg/kg, e.g., 50 mg/kg, e.g., at days −3, −2, −1 prior to transplantation). The antibody preparation eliminates mature T cells and natural killer cells. The ATG preparation also eliminates natural killer (NK) cells. Anti-human ATG obtained from any mammalian host can also be used. In addition, if further T cell depletion is indicated, the recipient may be treated with a monoclonal anti-human T cell antibody, such as LoCD2b (Immerge BioTherapeutics, Inc., Cambridge, Mass.). For bone marrow transplant, the recipient can be administered low dose radiation. In some cases, the recipient can be treated with an agent that depletes complements, such as cobra venom factor (e.g., at day −1).

In some embodiments, maintenance therapy (e.g., beginning immediately prior to, and continuing for at least a few days after transplantation) includes treatment with a human anti-human CD154 mAb. Mycophenolate mofetil (MMF) may be administered to maintain the whole blood levels. Methylprednisolone may also be administered, beginning on the day of transplantation, tapering thereafter over the next 3-4 weeks.

Various agents useful for supportive therapy (e.g., at days 0-14) include anti-inflammatory agents such as prostacyclin, dopamine, ganiclovir, levofloxacin, cimetidine, heparin, antithrombin, erythropoietin, and aspirin.

In some embodiments, donor stromal tissue is administered. It may be obtained from fetal liver, thymus, and/or fetal spleen, may be implanted into the recipient, e.g., in the kidney capsule. Thymic tissue can be prepared for transplantation by implantation under the autologous kidney capsule for revascularization. Stem cell engraftment and hematopoiesis across disparate species barriers may be enhanced by providing a hematopoietic stromal environment from the donor species. The stromal matrix supplies species-specific factors that are required for interactions between hematopoietic cells and their stromal environment, such as hematopoietic growth factors, adhesion molecules, and their ligands.

As liver is the major site of hematopoiesis in the fetus, fetal liver can also serve as an alternative to bone marrow as a source of hematopoietic stem cells. Each organ includes an organ specific stromal matrix that can support differentiation of the respective undifferentiated stem cells implanted into the host. As an alternative or an adjunct to implantation, fetal liver cells can be administered in fluid suspension.

Bone marrow cells (BMC), or another source of hematopoietic stem cells, e.g., a fetal liver suspension, of the donor can be injected into the recipient. Donor BMC home to appropriate sites of the recipient and grow contiguously with remaining host cells and proliferate, forming a chimeric lymphohematopoietic population. By this process, newly forming B cells (and the antibodies they produce) are exposed to donor antigens, so that the transplant will be recognized as self. Tolerance to the donor is also observed at the T cell level in animals in which hematopoietic stem cell, e.g., BMC, engraftment has been achieved. The use of xenogeneic donors allows the possibility of using bone marrow cells and organs from the same animal, or from genetically matched animals.

### EXAMPLES

This invention will be better understood from the Experimental Details, which follow. However, one skilled in the art will readily appreciate that the specific methods and results discussed are merely illustrative of the invention as described more fully in the claims that follow thereafter.

### Example 1—Generation of Human/Pig Hybrid Thymus to Achieve Immune Tolerance to Pig Antigens with Optimal Immune Function

Powerful immune responses to xenografts are difficult to control with conventional immunosuppression without excessive toxicity. Thymus transplantation is a promising approach to induce T cell tolerance for xenotransplantation. It has previously been shown that humanized mice generated with human hematopoietic stem cells (HSCs) and swine (SW) thymus grafts are tolerant to both species. However, there are still several challenges to this approach. First, T cells selected on SW MHC in pig thymus may not optimally recognize antigens presented by human MHC (HLA) in the periphery. Secondly, as SW thymic epithelial cells (TECs) do not display human tissue-restricted antigens (TRAs), there might be impaired negative selection and also a lack of Tregs specific for human TRAs. These problems were overcome by creating a human/pig hybrid thymus described herein.

### Methods

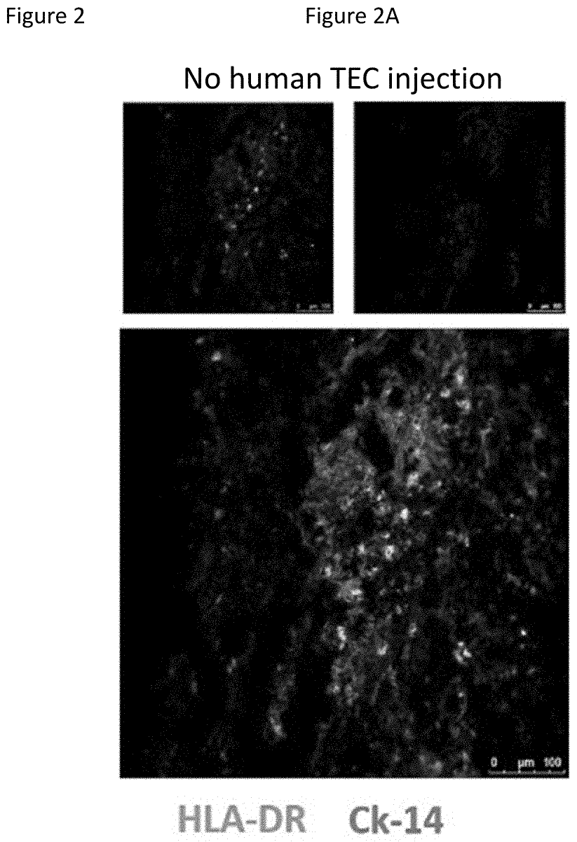

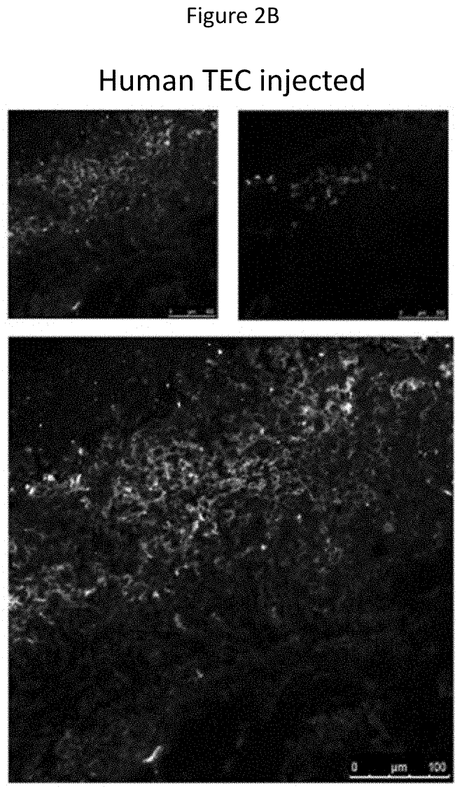

In order to generate human/pig hybrid thymi, thymic stromal cells were isolated by digestion of human fetal (gestational age 20 weeks) and pediatric (4 month old) thymi with liberase followed by magnetic depletion of human CD45+ cells. The human CD45− cells were resuspended in Matrigel and injected into freeze/thawed fetal SW thymic tissue that had been treated with 2-deoxyglucose, which suppresses glycolysis, to reduce the numbers of pig thymocytes in the fetal SW thymus (FIGS. 2A-C).

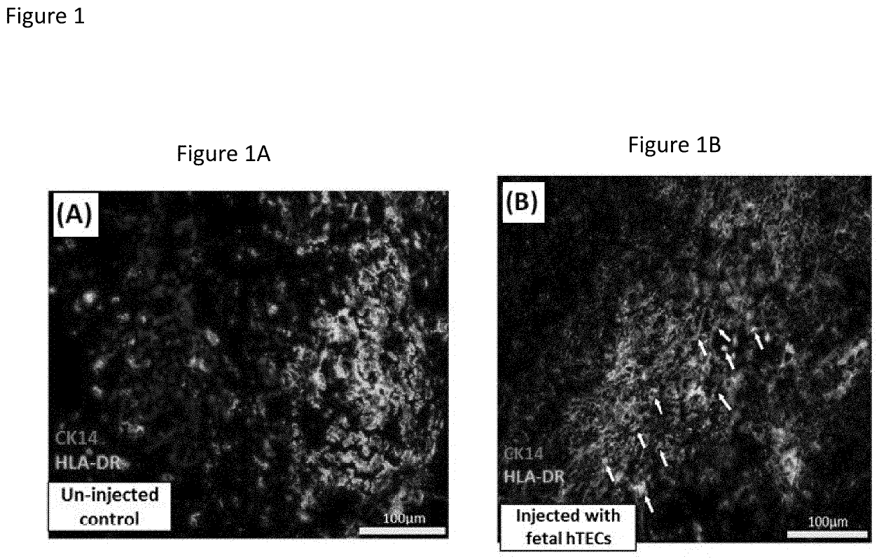

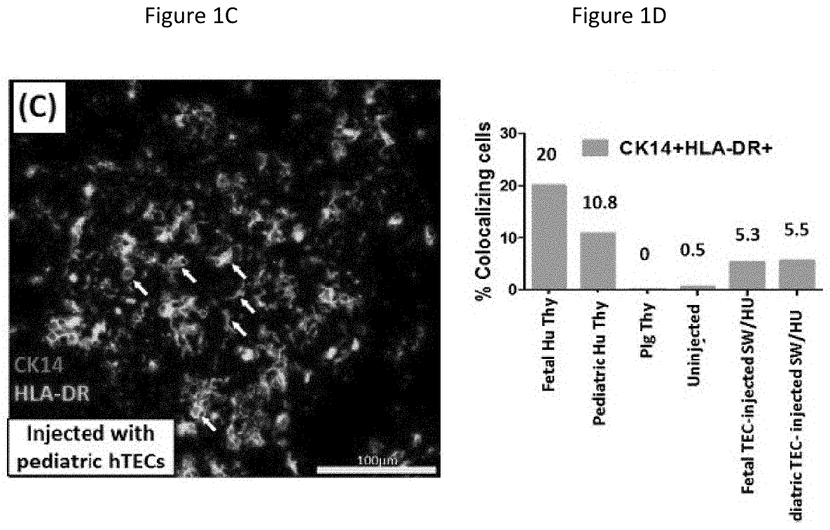

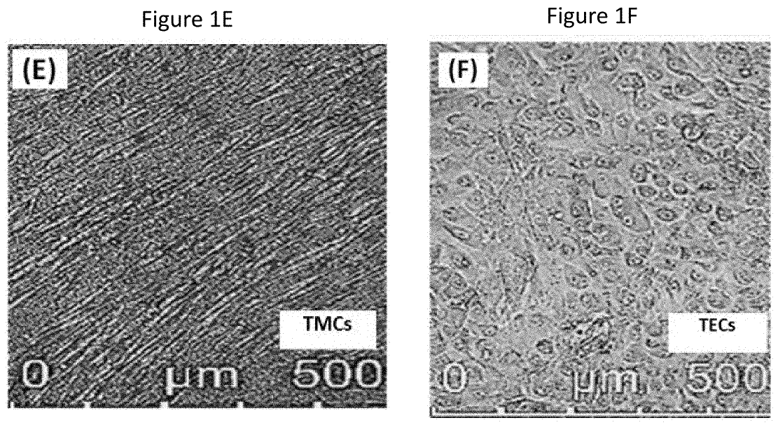

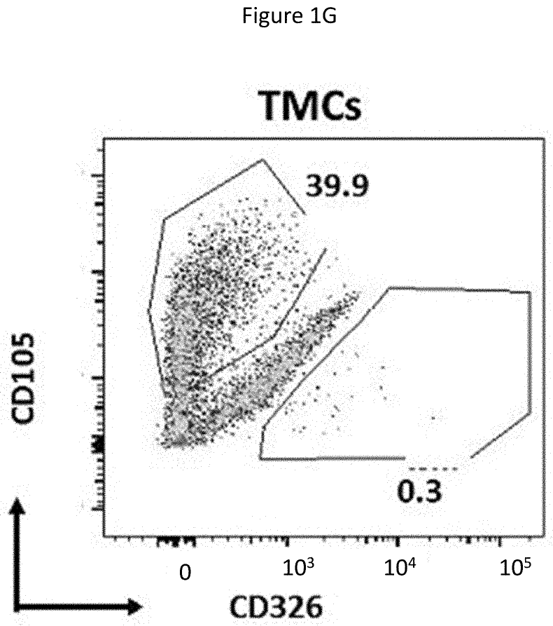

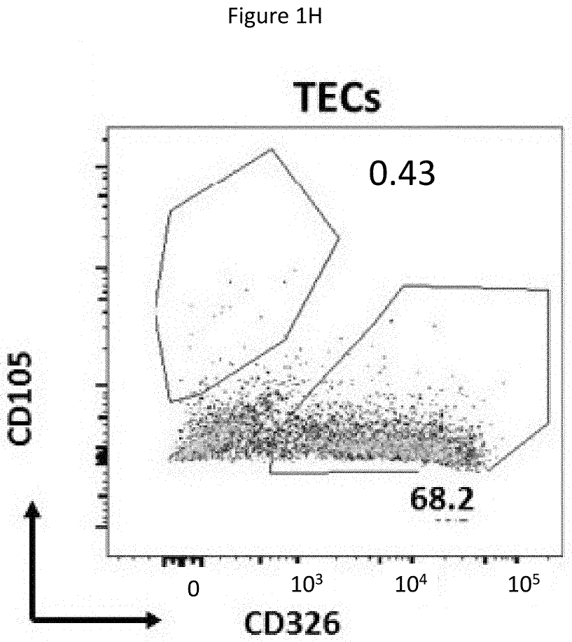

These injected SW thymi were then implanted into irradiated NOD scid common y chain knockout (NSG) mice followed by injection of human fetal liver-derived CD34+ HSCs from the same huTEC donor or an allogeneic donor. At 12-20 weeks post-transplantation, the grafted thymi were removed, sectioned and stained to detect human TECs using two-photon confocal microscopy (FIGS. 1A-1D). As the number of TECs in adult thymi decreased due to thymic involution, thymus-derived huTECs and thymic mesenchyme cells (TMCs) from a 17-year-old donor were expanded on a 2D Matrigel matrix and the cells injected into fetal swine thymic tissues, followed by transplantation to humanized mice (FIGS. 1E-1H).

### Results

HuTEC-injected SW thymi were functional and supported human thymopoiesis in humanized mice. Cytokeratin (CK) 14+ HLA-DR+ cells and CK8+ HLA-DR+ cells as well as CK8+ CK14+ HLA-DR+ cells were detected in hybrid thymi generated by both human fetal and pediatric donors. These TECs were distributed widely and admixed with pig TECs (FIGS. 1A-1D). 17-year-old thymus EpCAM+ TECs were expanded 5-fold in a single passage. Hybrid thymi that were generated with the in vitro-expanded human TECs and mesenchyme cells contained human TECs (FIGS. 1E-1H).

### CONCLUSION

Injection of human thymic stromal cells into pig thymus is an effective approach to generate human/pig hybrid thymus. Human TECs from older thymi can be expanded in vitro with the protocol described herein and were detectable long-term in pig grafts.

### Example 2—Development of the Method for Generating Hybrid Thymi

### Method of Injection

Cells were resuspended in Matrigel to prevent them from leaking out of the pig thymus after injection. As a proof of principle, in this experiment, human PBMCs were used instead of human thymic epithelial cells. First, 10 million human PBMCs were stained with CFSE (2.5 μM) as a tracing dye. CFSE-stained PBMCs (8 million cells) were resuspended in 140 μl Matrigel on ice with a cell concentration of 50,000 cells per μl. Three different methods for injection of the cells into the thawed swine fetal thymic fragments:
Method A: Injection using Hamilton syringe while the pieces were placed inside the wells of a V-bottom 96-well plate (5-8 μl);
Method B: Injection using PESO tubing (20 μl);
Method C: Injection using Hamilton syringe while the pieces were kept outside the well with forceps until the Matrigel is solidified (4-6 μl).

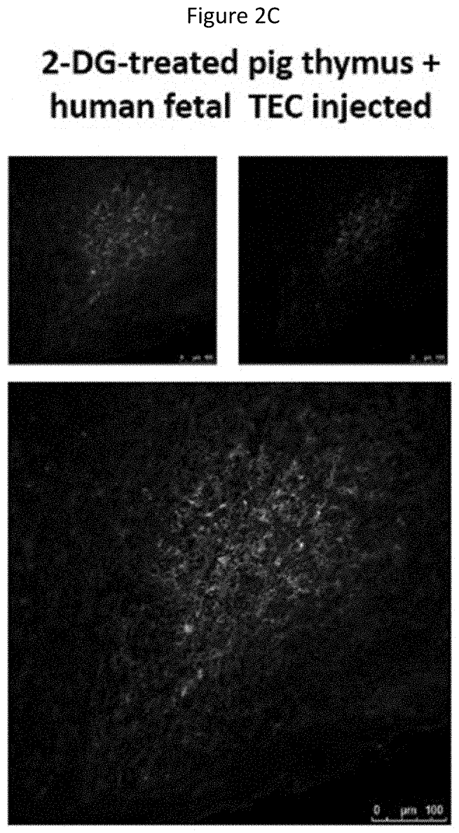

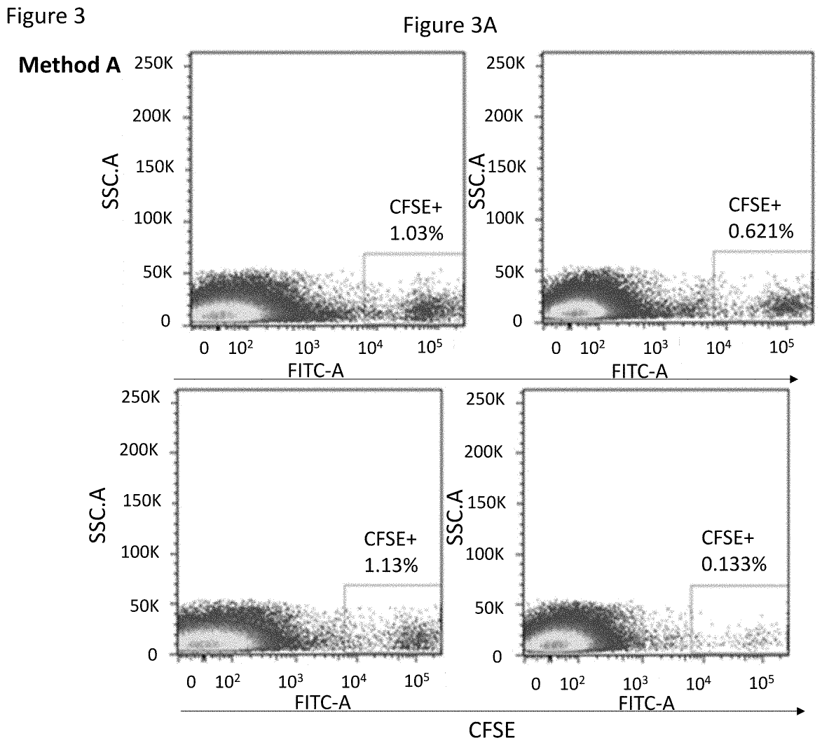

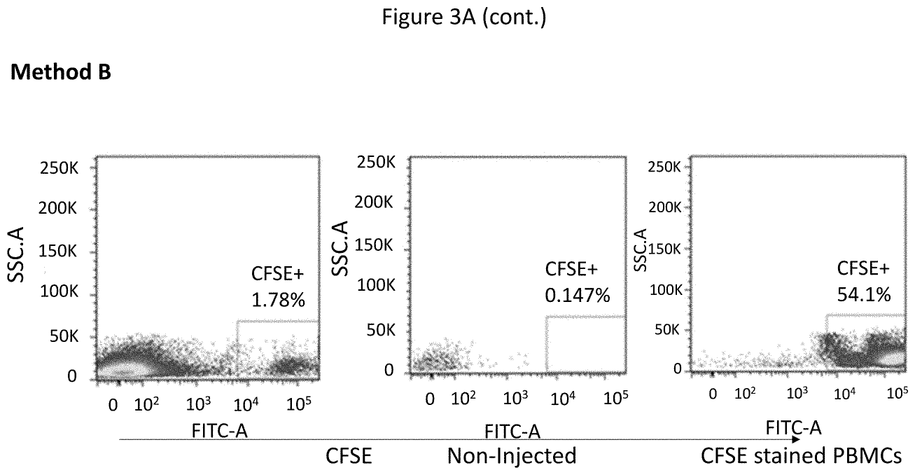

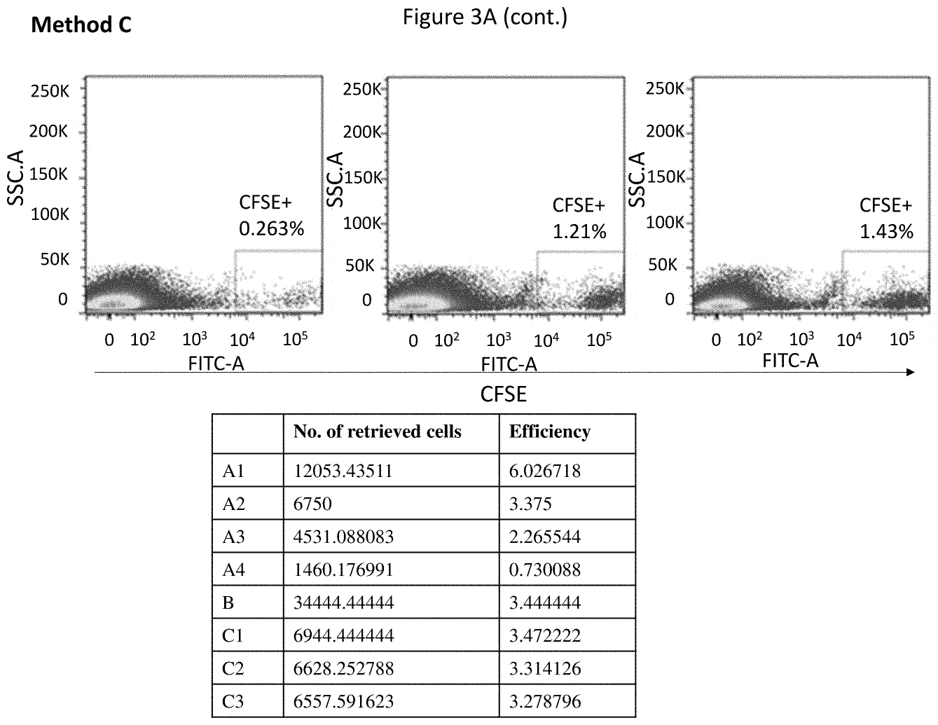

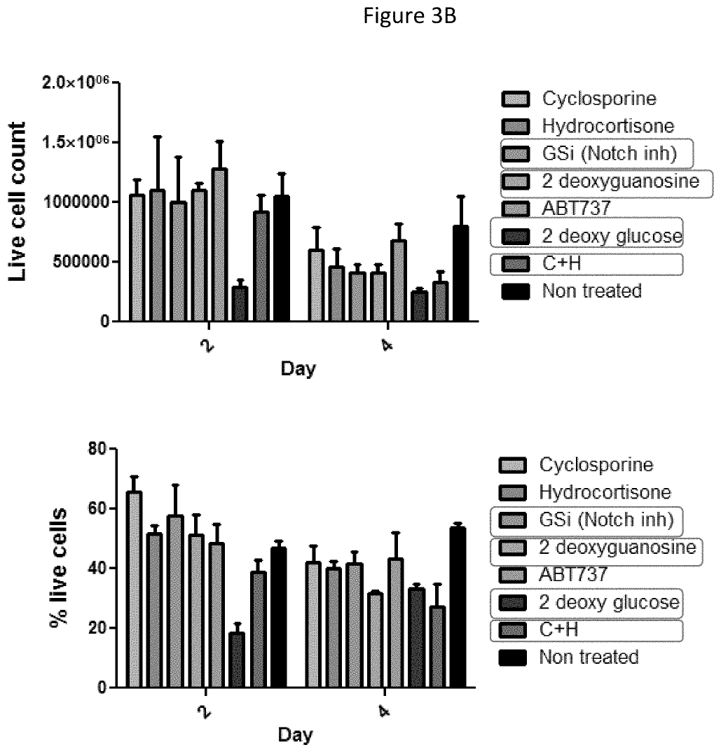

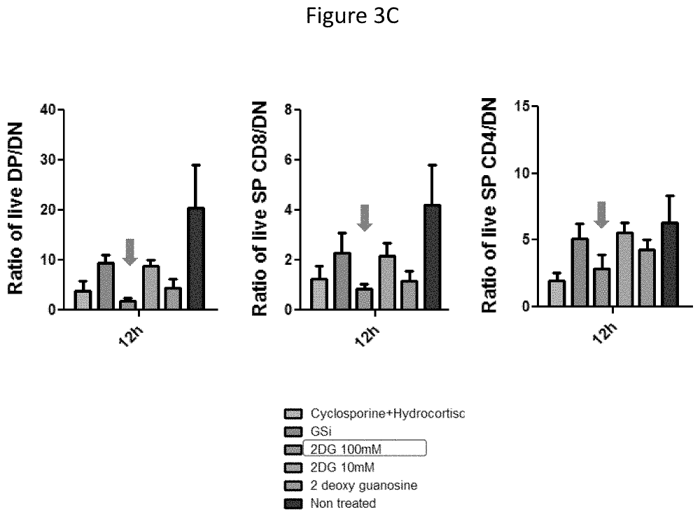

All the injected pieces were transferred to different wells of a 96-well plate containing DMEM/F12 medium supplemented with 10% FBS. After 3 hours, the pieces were digested with liberase. The number of released cells was determined by flow cytometry to track the CFSE-stained injected PBMCs. As shown in FIG. 3A, for all three injection methods, at least a portion of the cells that were injected were retrieved.

In summary, resuspension of the cells in Matrigel before injection helped retain the injected cells in the swine thymic tissue. Matrigel is the trade name for a gelatinous protein mixture secreted by Engelbreth-Holm-Swarm (EHS) mouse sarcoma cells produced and marketed by Corning Life Sciences and BD Biosciences.

### Method of Depletion of Pig Thymocytes to Prevent Rejection of Human Thymic Stromal Cells

There are different strategies to deplete the thymocytes in pig thymic fragments ex vivo, including anti-pig CD3 immunotoxin and complement-mediated toxicity (rabbit serum as source of complements plus anti-pig CD2). However, these two strategies were not effective. The former strategy induced apoptosis in pig thymocytes at higher concentrations, killed SP and DP T cells as well as non-T cells, and left a large population of live thymocytes in the thymus. The latter strategy was not effective in depleting pig thymocytes in thymic fragments ex vivo. Results not shown.

In view of these results, the following reagents were tested for depleting pig thymocytes ex vivo using the following method:
Day 0: Thawing pig thymus pieces, incubate them with one of the following (in triplicate):
a) Cyclosporine A
b) Hydrocortisone
c) Notch inhibitors (Gamma secretase inhibitors=GSi)
d) ABT-737
e) 2 deoxyguanosine
f) 2D Glucose
g) No treatment
   At different time points, the tissues were digested and tested for dead and apoptotic cell percentages. See FIG. 3B.

Ex vivo conditioning with 2-deoxyglucose (2DG, 100 mM) for 12 hours was the best approach to deplete the thymocytes, while keeping the stromal cells alive. The ratio of remaining live double positive CD4 and CD8 cells (DP) or single positive (SP) CD4 (SP-CD4) or SP-CD8 cells on double negative (DN) cells, which contain the stromal cells, was used as the readout. Treatment with 2DG 100 nM for 12 hours resulted in the lowest ratios and therefore was the best strategy for removing thymocytes while preserving the stromal cells. See FIG. 3C.

### Generation of the Hybrid Thymi

Using the 2DG treatment to deplete the pig thymocytes and the Hamilton syringe (Method C described above) for injection of human thymic stromal cells, a hybrid thymus was generated and tested in vivo. Specifically, two vials of fetal pig thymus were thawed. After pipetting up and down to release as many thymocytes as possible, half of the pieces were treated with 100 mM 2DG for 12 hours and the other half remained untreated.

On the next day, six vials of human fetal thymus were thawed. After pipetting up and down to release as many thymocytes as possible, liberase was used to digest and release the stromal cells and make a single-cell suspension. After depletion of human CD45+ cells using magnetic-activated cell sorting (MACS), 10 million thymic stromal cells were dissolved in 150 μl of cold Matrigel at a concentration of 66,000 cells per μl. Around 10 μl of the cells (around 660,000 thymic stromal cells) were injected into each fetal pig thymus piece (either 2DG-treated and non-treated). The pieces were kept with fine forceps at room temperature for about 2 minutes until the Matrigel solidified. Then, each piece was transferred to a well of a 96-well plate containing medium with 10% human serum. As a control, some pieces were not injected with human thymic stromal cells. After 10 minutes in the incubator, the plate was transferred on ice to the mouse facility for transplantation to immunodeficient NSG mice. The recipient mice were previously thymectomized, so they had no native mouse thymus and the only place for thymopoiesis was the grafted thymic fragments. The recipient NSG mice were first irradiated (100 cG), followed by injection of human fetal liver-derived CD34+ hematopoietic stem cells (HSCs). Then, one thymic piece was transplanted under the kidney capsule of each recipient mouse.

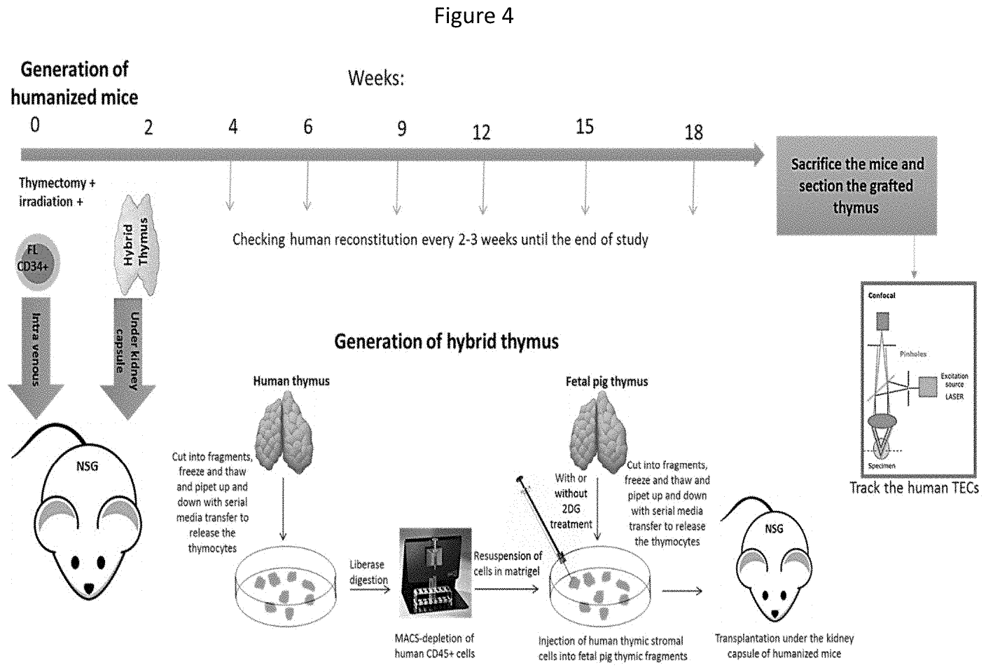

The experimental scheme is shown in FIG. 4.

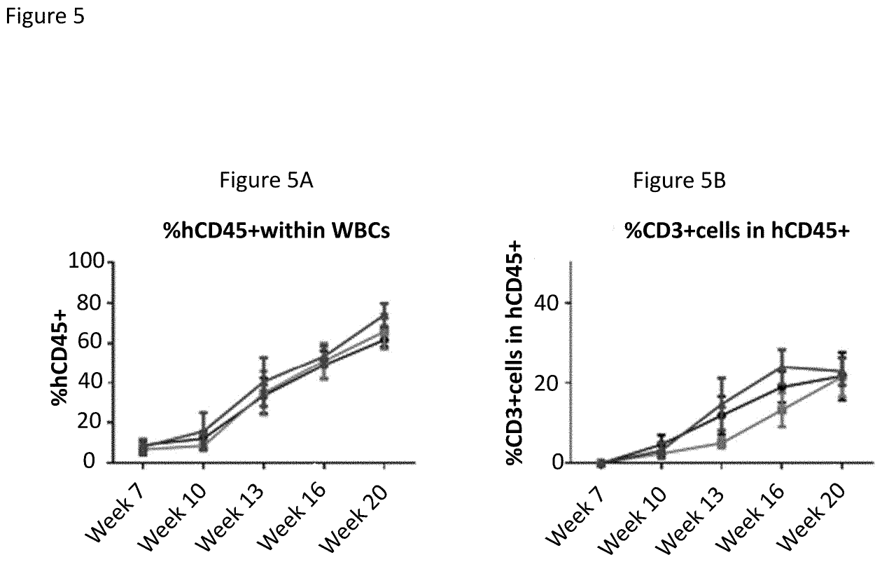

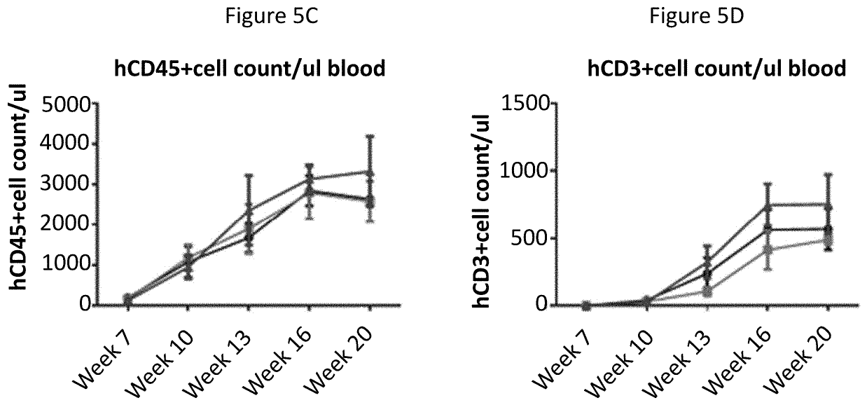

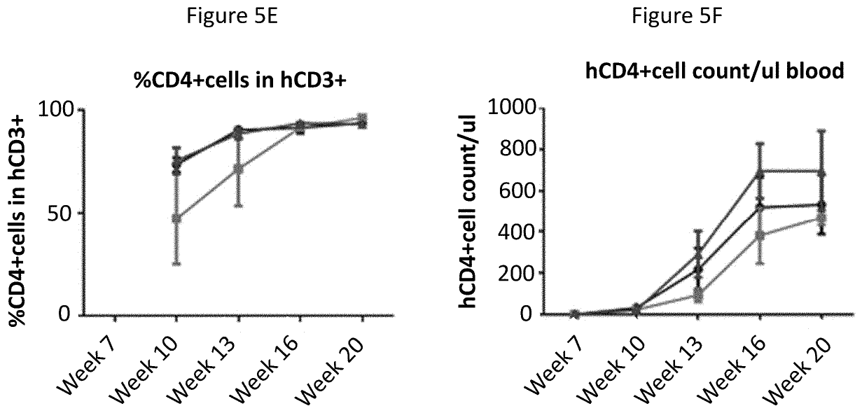

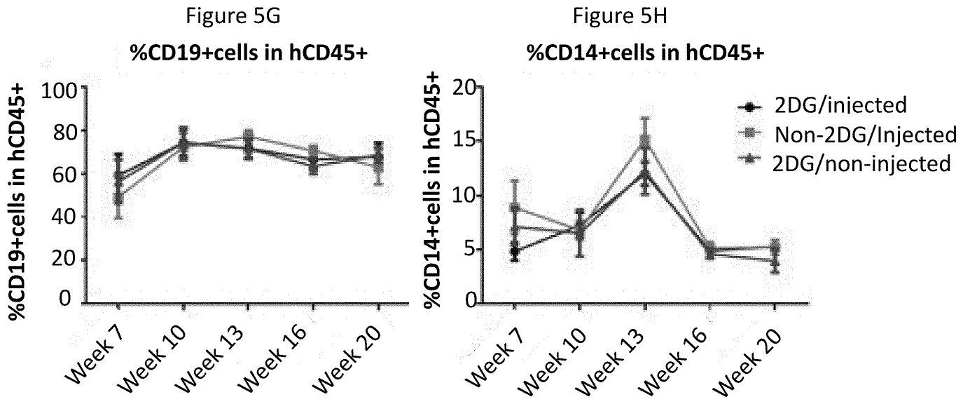

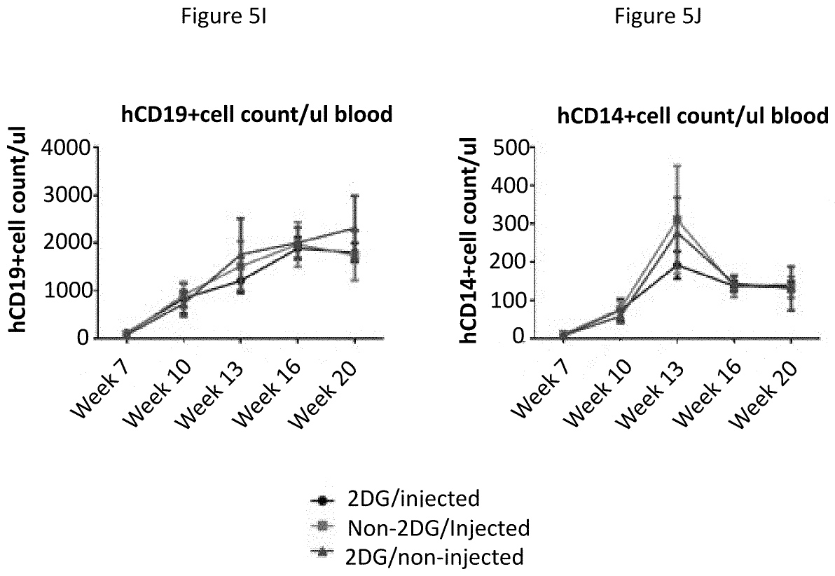

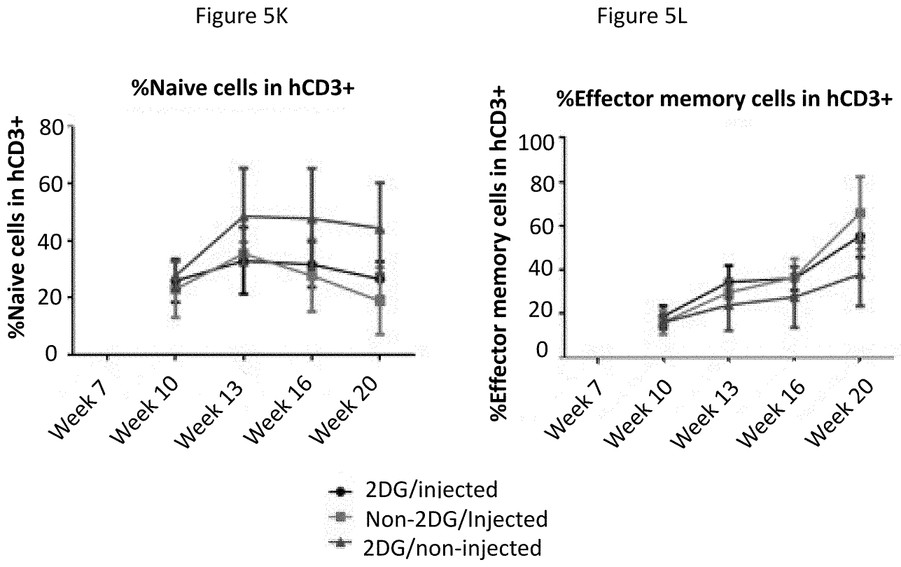

Every 2-3 weeks following transplantation, the mice were bled, and the level of human immune cell reconstitution was evaluated. As shown in FIG. 5, treatment of pig thymic fragments with 2DG did not affect human thymopoiesis in grafted thymi.

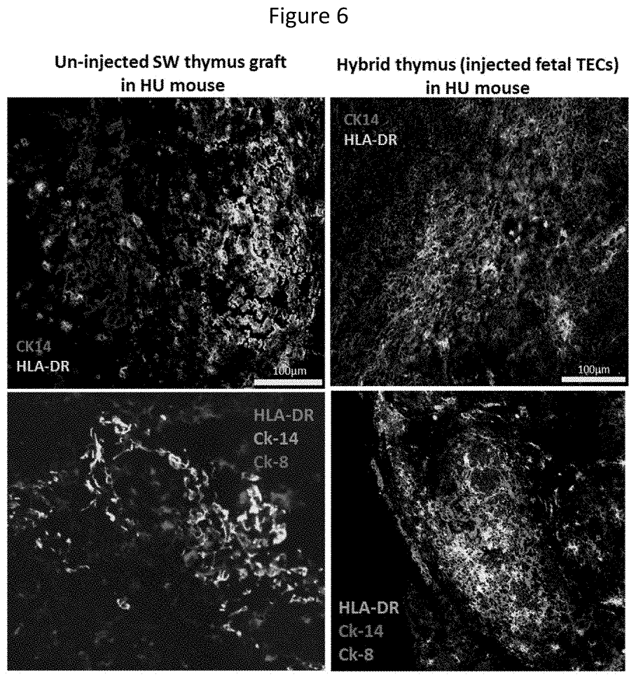

At 20 weeks post-transplantation, the mice were euthanized and the grafted thymi removed and froze in OCT. After cryosectioning, the slides were stained with antibodies against human HLA-DR, cytokeratin 8 (CK8, as a marker of cortical thymic epithelial cells (TECs)) and CK14 (as a marker of medullary TECs). As CK antibodies are cross-reactive for both human and pig, HLA-DR was used to differentiate human and pig TECs. As shown in FIG. 6, the injected human TECs admixed with pig TECs were detected within the grafted thymus. The HLA-DR+ cells that were negative for CKs were the HSC-derived antigen-presenting cells that migrated from bone marrow to the grafted thymi.

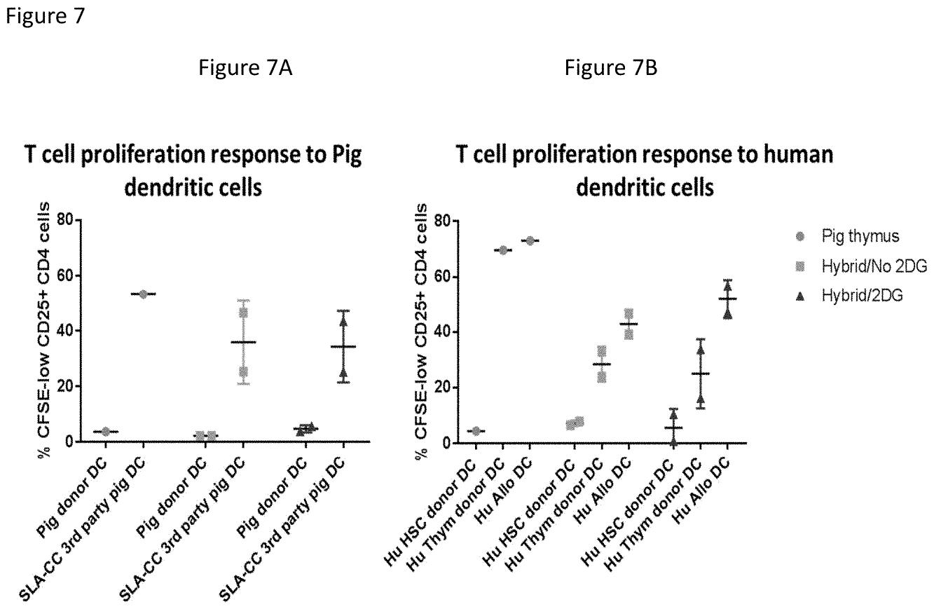

The in vitro T cell proliferation results suggested that peripheral T cells of mice with hybrid thymus were partially tolerant of the human TEC donor (FIG. 7B).

The human T cells from all mice were tolerant of pig donor dendritic cells (DCs), but not to the SLA-CC 3^{rd} party pig dendritic cells (DCs) (FIG. 7A).

The human T cells from all mice were tolerant of human HSC-donor DCs (FIG. 7B).

The human T cells from the mouse with a pig thymus proliferated at a similar level in response to both human thymus donor-DCs (allogeneic to the human HSCs) and human allo-DCs. Human T cells from the mice with hybrid thymus proliferated at a decreased level in response to human thymus donor-DCs compared to allo-DCs (FIG. 7B).

### Example 3—Increased Responsiveness to Human Tissue Restricted Antigens (TRAs) (MART-1, NYESO1 and Islet Antigen IA-2) Among Human T Cells that Develop in a Pig Thymus (SW/HU Mice) Compared to Those Developing in a Human Thymus (HU/HU Mice)

### Methods

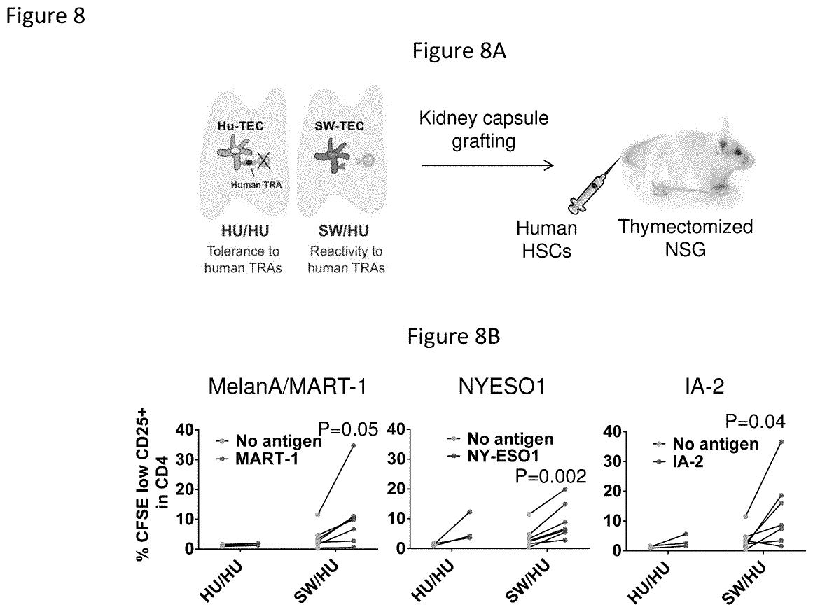

In order to evaluate the hypothesis that a lack of negative selection of human TRA-specific T cells developing in swine thymus, two groups of humanized mice were generated with the same human fetal liver CD34+ HSCs and either an autologous human fetal thymus (HU/HU) or a swine fetal thymus (SW/HU). See FIG. 8A. In both groups, the native mouse thymus was removed to insure that the thymopoiesis occurred only in either human or swine thymus. At about 22 weeks post-transplantation, the mice were euthanized and the pooled lymph node (LN) and spleen cells were depleted of mouse CD45+ cells using MACS. The remaining cells were co-cultured with autologous HSC-derived dendritic cells loaded with different human TRA proteins to measure the T cell proliferation in response to these TRAs.

### Results

As shown in FIG. 8B, human peripheral T cells in SW/HU mice showed significantly increased proliferative responses to human TRAs (IA-2, MART-1 and NYESO1) presented by autologous human DCs. The amino acid sequence of these TRAs is significantly different between human and pig. This finding supported the lack of negative selection of human TRA-specific T cells in swine thymus and demonstrated the need for using a hybrid thymus.

### Example 4—Lower Survival of Human Tregs and CD8 T Cells Develop in a Pig Thymus (SW/HU Mice) Compared to Those Developing in a Human Thymus (HU/HU Mice)

### Methods

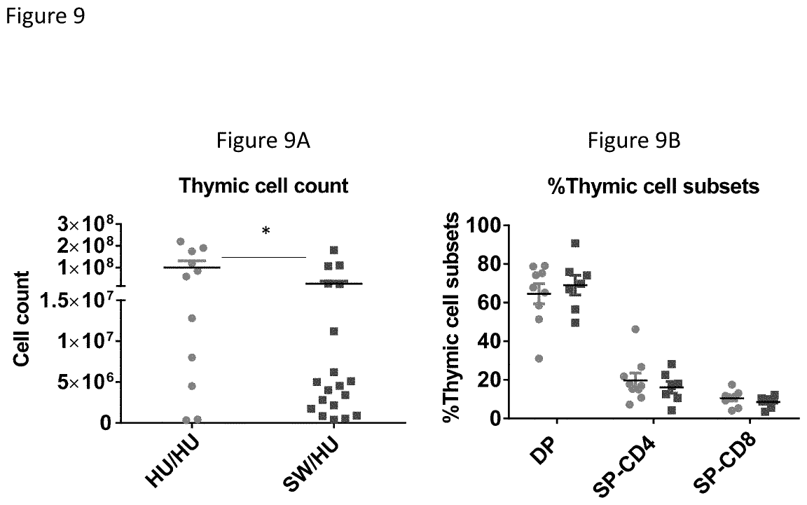

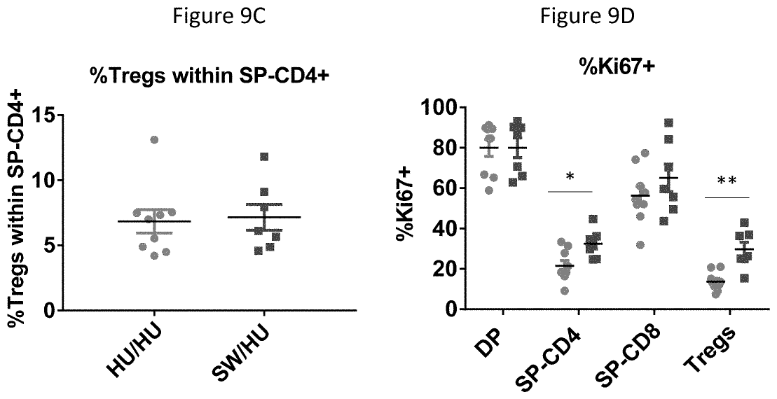

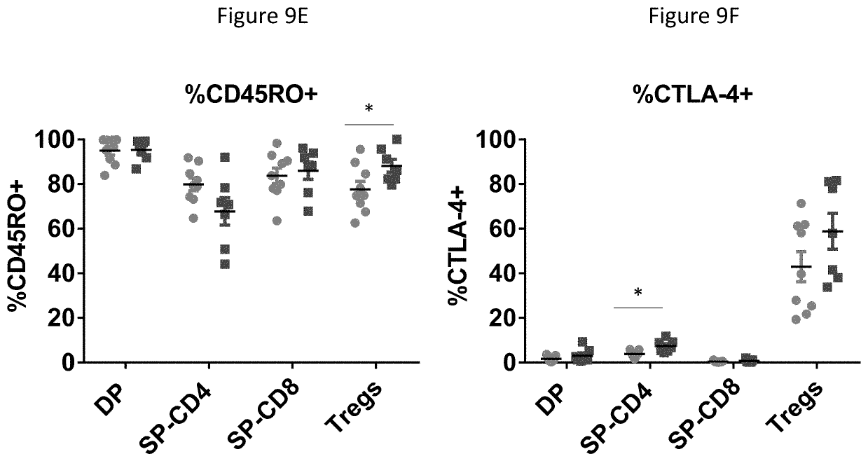

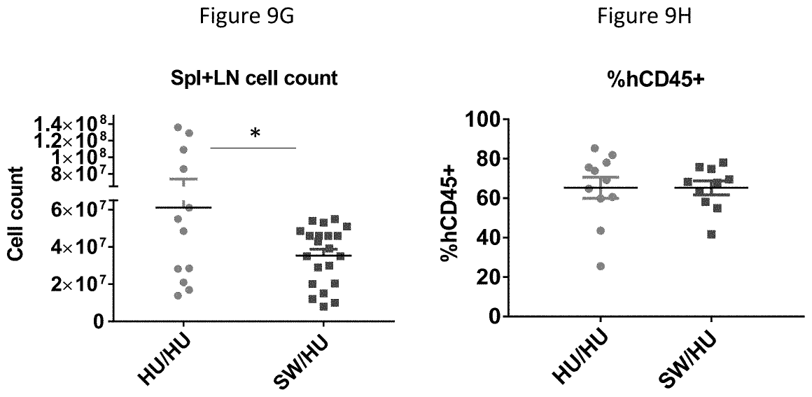

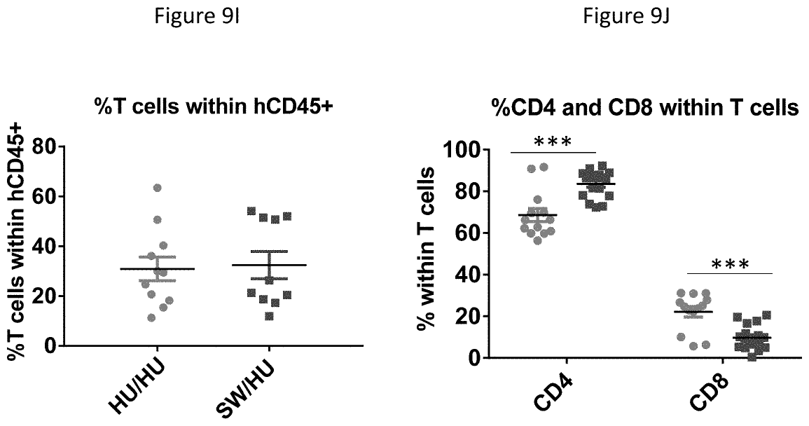

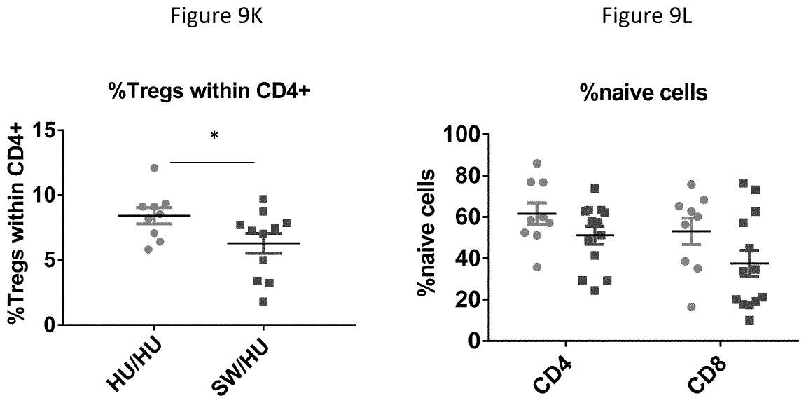

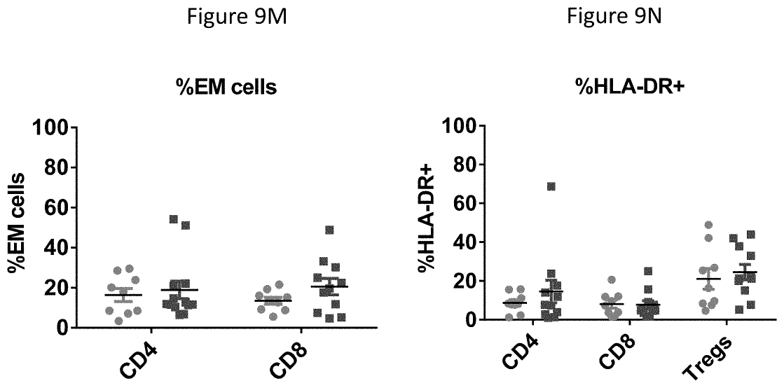

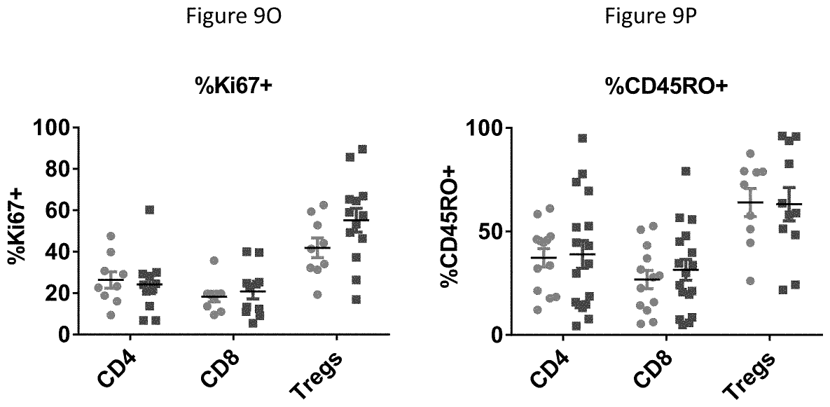

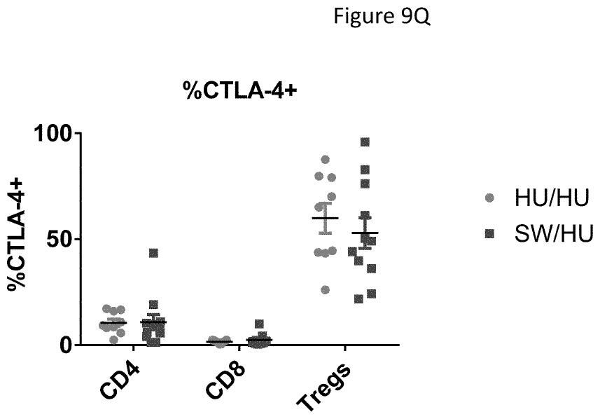

The mice generated in Example 3 (SW/HU and HU/HU mice) were euthanized at about 24 weeks post-transplantation. Grafted thymi and pooled spleen and lymph nodes (cervical, axillary, brachial and mesenteric LNs), were harvested. Thymocytes, spleen and LN cells were isolated by physical force (crushing the thymus tissue between two slides and crushing the spleen and LNs through a 70 μm cell strainer using a syringe plunger). ACK lysis buffer (Gibco) was used to lyse RBCs in spleen cells. Isolates cells were counted using a hemocytometer. After counting the total number of cells, 0.2-1 million cells from each thymus and pooled spleen and LNs were stained with the antibodies shown in FIG. 9 for flow cytometry analysis. Cells were read with a BD Fortessa flowcytometry machine and data were analysed with Flowjo software.

### Results

The number of spleen and LN cells as well as grafted thymic cells were significantly higher in HU/HU mice compared to SW/HU mice (FIGS. 9A and 9G). The ratios of DP (double positive CD4+CD8+) and SP cells (single positive either SP-CD4 or SP-CD8) were similar in the grafted thymi between the HU/HU and SW/HU mice (FIG. 9B). A functional thymus should have a higher ratio of DP than SP cells.

Also, the fraction of Tregs (regulatory T cells) in SP-CD4 cells were similar (FIG. 9C). The level of proliferating (Ki67+) SP-CD4 and Treg cells were higher in SW/HU thymi compared to HU/HU ones (FIGS. 9D and 9F). Also, higher fractions of SW/HU thymic Tregs expressed CD45RO (FIG. 9E).

Contrary to similar levels of SP-CD4 and SP-CD8 cells in thymus, SW/HU mice had lower proportions of CD8 cells among T cells and also Tregs among CD4 cells in the periphery (spleen and lymph nodes, FIGS. 9I-9K). This finding showed that both these cell subsets need to interact with the same MHC that they have been selected on for their survival. It appeared that there was a higher rate of naïve to memory conversion in both CD4 and CD8 cell subsets in the periphery of SW/HU mice (FIGS. 9L and 9M). The proportions of Ki67+ (proliferating) (FIG. 9O), HLA-DR+(activated) (FIG. 9N), CD45RO+ (FIG. 9P), and CTLA-4+ cells (FIG. 9Q) within peripheral CD4, CD8 and Treg cells were not different between HU/HU and SW/HU.

These results further demonstrated the need for using a hybrid thymus.

### Example 5—Generation of Hybrid Thymus with Embryonic Stem Cell-Derived TECs (ES-TECs) and Long-Term Persistence of Human TECs in the Hybrid Thymus

### Methods

Human fetal or pediatric thymic stromal cells (hu-TECs) or hPSC-TEC progenitors (1-2×10⁵ TECs) were injected into fetal pig thymus tissue (SW THY) prior to implantation under the kidney capsule of thymonectisized, immunodeficient NSG (Nod/Scid/Ilr2g−/−) mice, that had been injected intravenously with 2×10⁵ human fetal liver-derived CD34+ cells. Controls were implanted with fetal pig thymus tissue not injected with human TECs.

At about 20 weeks post-transplantation, the grafted thymi were removed, sectioned, and stained to detect human TECs using two-photon confocal microscopy.

The cells were then released from the stroma and the number of cells determined by flow cytometry.

### Results

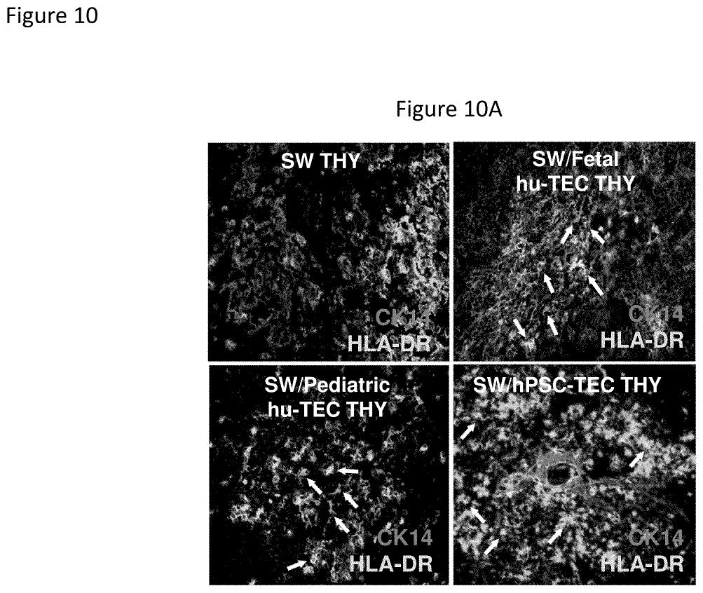

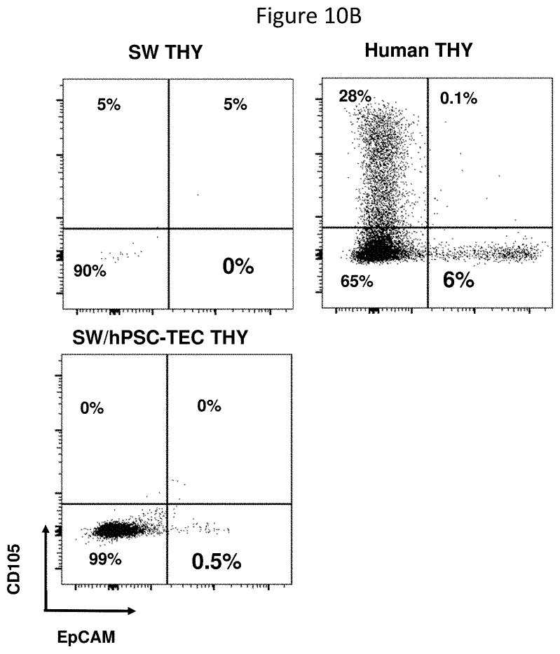

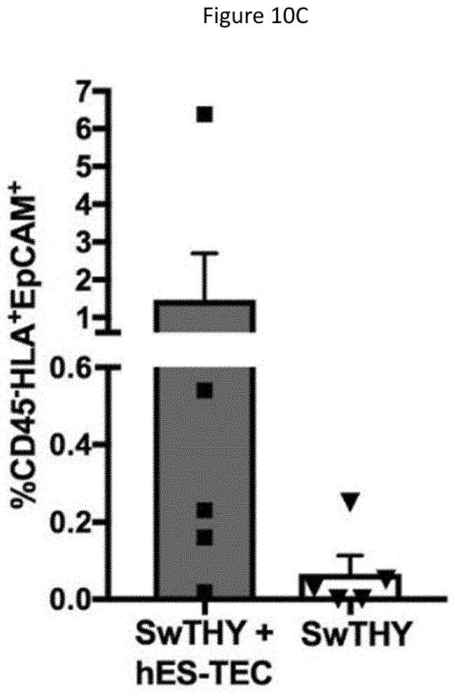

As shown in FIG. 10, there was a long-term (greater than 20 weeks) persistence of human TECs in “hybrid thymus”. As shown in FIG. 10A, all of the grafts, including the pig thymus not injected with human TECs (top left panel) have human HSC-derived HLA-DR+ APCs (green) and pig/human CK14+ TECs (red). However, only hybrid thymi have detectable HLA-DR+CK14+ human TECs (yellow color, see arrows).

Flow cytometric staining of gated CD45-negative cells in digested stroma from long-term thymic grafts showed the presence of EPCAM+, CD105-negative hu-TECs only in the human thymus (FIG. 10B, top right) and SW grafts that had been injected with hPSC-TEC progenitors (FIG. 10B, bottom left panel) but not the uninjected SW THY grafts (FIG. 10B, top left). See also FIG. 10C.

### Example 6—Injection of hES-TECs into Swine Thymus Promoted Increased T Cell Development and Increased Peripheral CD4⁺ and CD8⁺ T Cells

### Methods

hES-TEC (1-2×10⁵ TECs) were injected into fetal pig thymus tissue (SW THY) prior to implantation under the kidney capsule of thymectomized immunodeficient NSG (Nod/Scid/Ilr2g−/−) mice, that had been injected intravenously with 2×10⁵ human fetal liver-derived CD34+ cells. Controls were implanted with fetal pig thymus tissue not injected with human TECs.

Splenocytes and thymocytes from thymic grafts were analyzed by flow cytometry 18-22 weeks post-transplant.

### Results

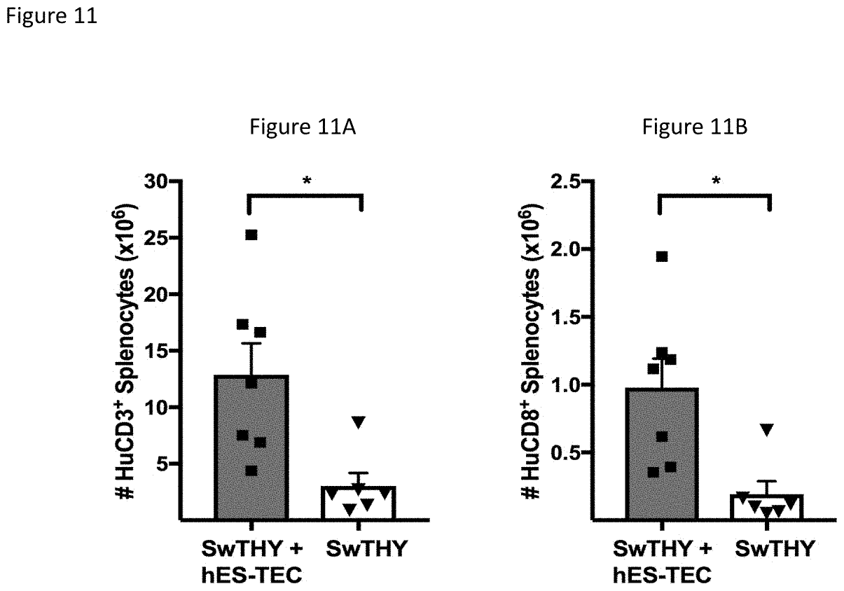

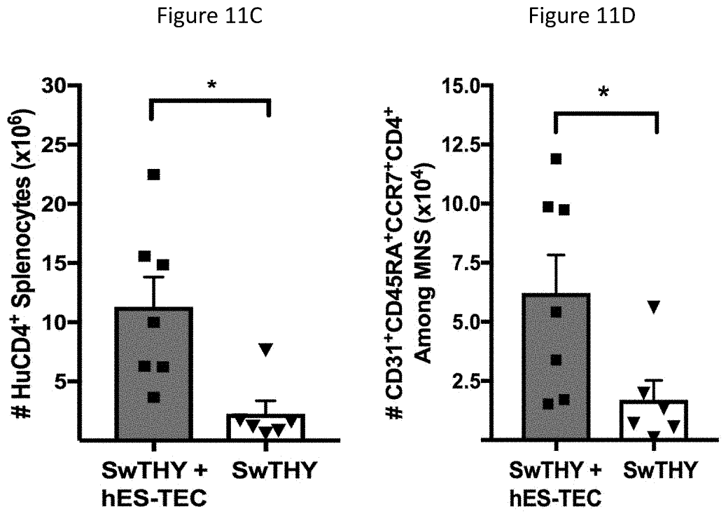

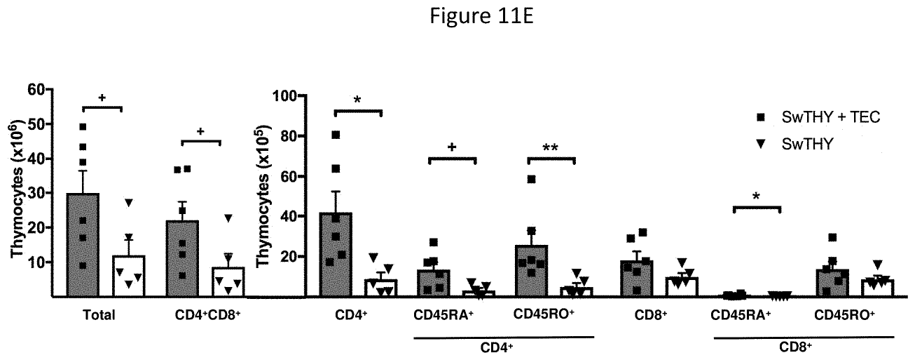

As shown in FIGS. 11A-D, the thymic grafts injected with the hES-TECs had higher absolute numbers of human splenic CD3⁺ T cells, CD8⁺ T cells, CD4⁺ T cells, and recent thymic emmigrant CD31⁺CD4⁺ naive cells defined as CD45RA⁺CCR7⁺ cells in mononuclear cells of the spleen.

To assess terminal stages of terminal differentiation, thymocytes were stained for expression of HuCD45, CD19, CD14, CD4, CD8, CD45RA and CD45RO. As shown in FIG. 11E, the thymic grafts injected with the hES-TECs had higher numbers of total thymocytes as well as higher numbers of human CD45 cells, double positive CD4+CD8+, single positive CD4+CD8-, CD4-CD8+, and immature CD45RO+.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

What is claimed is:

1. An isolated hybrid thymic tissue comprising thymic epithelial cells from a first mammalian species and a thymic tissue from a second mammalian species.

2. The isolated hybrid thymic tissue of claim 1, wherein the second species is swine.

3. The isolated hybrid thymic tissue of claim 2, wherein the swine is a miniature swine.

4. The isolated hybrid thymic tissue of claim 1, wherein the first species is primate or a human.

5. The isolated hybrid thymic tissue of claim 1, wherein the thymic tissue from the second mammalian species is a fetal thymic tissue or neonatal thymic tissue.

6. The isolated hybrid thymic tissue of claim 1, wherein the thymic epithelial cells are obtained from the first mammalian species thymus.

7. The isolated hybrid thymic tissue of claim 1, wherein the thymic epithelial cells are generated from induced pluripotent stem cells (iPSCs) or embryonic stem cells.

8. The isolated hybrid thymic tissue of claim 7, wherein the thymic epithelial cells are generated from embryonic stem cells that share HLA alleles with first mammalian species.

9. The isolated hybrid thymic tissue of claim 7, wherein the embryonic stem cells are genetically engineered to share HLA alleles with the first mammalian species.

10. The isolated hybrid thymic tissue of claim 1, wherein the hybrid thymic tissue is generated by introducing thymic epithelial cells from the first species into the thymic tissue from the second species.

11. The isolated hybrid thymic tissue of claim 1, wherein the hybrid thymic tissue is generated by injecting thymic epithelial cells from the first species into the thymic tissue from the second species.

12. The isolated hybrid thymic tissue of claim 1, wherein the hybrid thymic tissue is generated by a method comprising the following steps:
(i) treating the thymic tissue from the second species with 2-deoxyglucose (2DG); and
(ii) introducing thymic epithelial cells from the first species into the 2DG-treated thymic tissue.

13. The isolated hybrid thymic tissue of claim 12, wherein in step (ii) the thymic epithelial cells are suspended in Matrigel before being injected to the 2DG-treated thymic tissue.

14. A method for making a hybrid thymic tissue, comprising the steps of:
(i) treating a thymic tissue from a second mammalian species with 2-deoxyglucose (2DG); and
(ii) introducing thymic epithelial cells from a first mammalian species into the 2DG-treated thymic tissue.

15. The method of claim 14, wherein in step (ii) the thymic epithelial cells are suspended in Matrigel before being injected to the 2DG-treated thymic tissue.

16. The method of claim 14, wherein the second species is swine.

17. The method of claim 14, wherein the swine is a miniature swine.

18. The method of claim 14, wherein the first species is primate or a human.

19. The method of claim 14, wherein the thymic tissue is a fetal thymic tissue or neonatal thymic tissue.

20. The method of claim 14, wherein the thymic epithelial cells are obtained from a thymus of the first mammalian species.

21. The method of claim 14, wherein the thymic epithelial cells are generated from induced pluripotent stem cells (iPSCs) of the first mammalian species or from embryonic stem cells of the first mammalian species.

22. The method of claim 21, wherein the thymic epithelial cells are generated from embryonic stem cells that share HLA alleles with the first mammalian species.

23. The method of claim 22, wherein the embryonic stem cells are genetically engineered to share HLA alleles with the first mammalian species.

24. The method of claim 14, wherein the hybrid thymic tissue is generated by introducing thymic epithelial cells from the first species into the thymic tissue from the second species.

25. The method of claim 14, wherein the hybrid thymic tissue is generated by injecting thymic epithelial cells from the first species into the thymic tissue from the second species.

26. A method of inducing tolerance in a recipient mammal of a first species to a graft obtained from a donor mammal of a second species, the method comprising the steps of:
(a) introducing the hybrid thymic tissue of claim 1 into the recipient mammal; and
(b) implanting the graft from the donor mammal in the recipient mammal.

27. A method of restoring or inducing immunocompetence in a recipient mammal of a first species, the method comprising the step of introducing the hybrid thymic tissue of claim 1 into the recipient mammal.

28. A method of restoring or promoting thymus-dependent ability for T cell progenitors to develop into mature functional T cells in a recipient mammal of a first species, the method comprising introducing the hybrid thymic tissue of claim 1 into the recipient mammal.
